(19) United States
(12) Patent Application Publication
(10) Pub. No.: US 2026/0157892 A1
(43) Pub. Date: Jun. 11, 2026

(54) LEG GASKETING SYSTEM FOR ABSORBENT ARTICLES

(71) Applicant: The Procter & Gamble Company, Cincinnati, OH (US)

(72) Inventors: Abhishek SURUSHE, Mason, OH (US); Gavin Kahlil DILLAVOU-BROWN, Cincinnati, OH (US); Koichi MORIMOTO, Cincinnati, OH (US); Kaoru ISHIHARA, West Chester, OH (US); Aaron Jeffrey KNOOP, Mason, OH (US); Christopher Erin KIGER, Union Township, OH (US); Keith Richard WILLHAUS, Cincinnati, OH (US)

(21) Appl. No.: 19/180,260

(22) Filed: Apr. 16, 2025

Related U.S. Application Data
(60) Provisional application No. 63/637,486, filed on Apr. 23, 2024.

(51) Int. Cl. A61F 13/494; A61F 13/49

(52) U.S. Cl. CPC A61F 13/49406; A61F 13/49017; A61F 2013/49025; A61F 2013/4948

(57) ABSTRACT

Aspects of the present disclosure relate to absorbent articles including a leg gasketing system comprising a first barrier cuff and a second barrier cuff laterally separated from the first barrier cuff with features adapted for mitigation of leakage and wearer entanglement while donning. As discussed in more detail below, the first barrier cuff and/or the second barrier cuff may be bonded with the chassis in proximal bond regions configured to allow for increased cuff heights while at the same time helping to mitigate wearer entanglement during donning. In addition, the first barrier cuff and/or the second barrier cuff may be bonded with the chassis in end bond regions configured to provide void regions between the barrier cuffs and the chassis in waist end regions to help prevent urine from leaking outside the article while the urine is being absorbed by the absorbent core.

### CROSS REFERENCE TO RELATED APPLICATION

This application claims the benefit of U.S. Provisional Application No. 63/637,486, filed Apr. 23, 2024, which is incorporated by reference herein in its entirety.

### FIELD

The present disclosure relates to absorbent articles, and more particularly, to absorbent articles including a leg gasketing system with features adapted for mitigation of leakage and wearer entanglement while donning.

### BACKGROUND

Various types of articles, such as for example, diapers and other absorbent articles may be adapted to absorb and retain soils and bodily waste. In turn, absorbent articles may include an absorbent chassis including features such as an absorbent core and/or acquisition/distribution system. The absorbent core and/or acquisition/distribution system may be sandwiched between a liquid pervious topsheet and a liquid impervious backsheet. Some absorbent articles may be configured to place the absorbent core and acquisition/distribution system in close proximity to a wearer's urine exit point (also referred to a “pee point”). Such an arrangement may help the absorbent article to acquire and distribute liquid bodily exudates, such as urine, around the pee point where liquid is initially introduced into the absorbent article. Various articles may be designed with component placements to function with a pee point that is positioned in a generally laterally central location on the absorbent article.

In addition, to further enhance performance, some absorbent articles may include a leg gasketing system. Such leg gasketing systems may include an inner leg cuff and an outer leg cuff. In some configurations, the inner leg cuff may be adapted to help prevent leakage of bodily exudates and the outer leg cuff may be adapted to help provide a covering to minimize the visibility of exudates through the inner leg cuff and to provide a secondary means to capture bodily exudates should they breach the inner leg cuff. The inner leg cuff may be made from a substantially liquid impervious nonwoven material and may be disposed on a body-facing surface of an absorbent article to provide a sealing directly against a wearer's skin. The inner leg cuff may be connected with the absorbent chassis and may extend for a height above and across the topsheet to a free edge. The outer leg cuff may also be made from a substantially liquid impervious nonwoven material and may be disposed laterally outside the inner leg cuff. The inner leg cuff and/or the outer leg cuff may include elastic elements extending in a longitudinal direction. Forces exerted by the elastic elements may help the surfaces of the inner leg cuff and/or outer leg cuff against a wearer's skin during wear.

Some absorbent articles are designed to fit relatively small wearers, such as infants and toddlers. In addition, some absorbent articles may be adapted to fit relatively larger wearers, such as children in later stages of toilet training. Such relatively large absorbent articles may be particularly useful to help prevent wetting of clothing and bedding while a child is sleeping. However, rather than sleeping on one's back or stomach, some relatively larger wearers of absorbent articles may have a tendency to sleep their sides. As such, a wearer that sleeps on their side may cause the pee point to shift laterally from a central location of the absorbent article to a side location of the absorbent article. Such lateral shifting of the pee point from the center of the absorbent article may result in unintended leakage. To help mitigate such unintended leakage, the heights of the inner leg cuffs may be increased. However, increased inner leg cuff heights may cause other unintended problems. For example, a wearer's feet may be more likely to become entangled with relatively high inner leg cuffs when donning the absorbent article. In addition, relatively high inner leg cuffs results in reduced spacing between laterally separated inner leg cuffs, and as such, may reduce an overall area of the absorbent core that is exposed to a wearer.

Consequently, it would be beneficial to provide absorbent articles with leg gasketing systems adapted for relatively large wearers to help mitigate urine leakage when such wearers may be prone sleeping on their sides while at the same time helping to reduce the likelihood of wearer interference during article donning.

### SUMMARY

In one form, an absorbent article comprises: a first waist region and a second waist region longitudinally separated from the first waist region by a crotch region; a chassis comprising a topsheet, a backsheet; and an absorbent core positioned between the topsheet and the backsheet, wherein the chassis further comprises a first lateral end edge in the first waist region longitudinally separated from a second lateral end edge in second waist region and comprising a longitudinal length CL of from about 400 mm to about 600 mm extending between the first lateral end edge and the second lateral end edge, and wherein the chassis further comprises a first longitudinal side edge laterally separated from a second longitudinal side edge; a leg gasketing system comprising a first barrier cuff and a second barrier cuff laterally separated from the first barrier cuff; a proximal bond region connecting the first barrier cuff with the chassis adjacent the first longitudinal side edge, the proximal bond region extending longitudinally for substantially the same length CL as the chassis, the first barrier cuff extending laterally inward toward the second barrier cuff from the proximal bond region to a distal edge; a first end bond region adjacent the first lateral end edge of the chassis, the first end bond region connecting the first barrier cuff with the chassis, wherein the first end bond region extends longitudinally inward from the first lateral end edge and extends laterally inward from the proximal bond region toward the distal edge to define a first void region where the first barrier cuff is not bonded with the chassis between the first end bond region and the proximal bond region, wherein the first void region defines a first void volume adapted to accept bodily exudates; a second end bond region adjacent the second lateral end edge of the chassis, the second end bond region connecting the first barrier cuff with the chassis, wherein second end bond region extends longitudinally inward from the second lateral end edge and extends laterally inward from the proximal bond region toward the distal edge to define a second void region where the first barrier cuff is not bonded with the topsheet between the second end bond region and the proximal bond region, wherein the second void region defines a second void volume adapted to accept bodily exudates; and wherein the first barrier cuff comprises a central segment extending longitudinally between a first segment and a second segment, the first segment extending longitudinally between the first end bond region and the central segment, and the second segment extending longitudinally between the second end bond region and the central segment, wherein the first barrier cuff is not bonded with the chassis between the proximal bond region and the distal edge in the first segment, the central segment, and the second segment, and wherein the first segment of the first barrier cuff comprises: a first cuff height defined by a distance between the proximal bond region and the distal edge, the second segment comprises a second cuff height defined by a distance between the proximal bond region and the distal edge, and the central segment comprises a central cuff height defined by a distance between the proximal bond region and the distal edge, and wherein the central cuff height is less than the first height and the second cuff height.

In another form, gender specific absorbent articles may comprise: a first absorbent article configured for a wearer of a first gender and a second absorbent article configured for a wearer of a second gender; wherein the first absorbent article and the second absorbent article each comprise: a first waist region and a second waist region longitudinally separated from the first waist region by a crotch region; a chassis comprising a topsheet, a backsheet; and an absorbent core positioned between the topsheet and the backsheet, wherein the chassis further comprises a first lateral end edge in the first waist region longitudinally separated from a second lateral end edge in second waist region and comprising a longitudinal length CL of from about 400 mm to about 600 mm extending between the first lateral end edge and the second lateral end edge, and wherein the chassis further comprises a first longitudinal side edge laterally separated from a second longitudinal side edge; a leg gasketing system comprising a first barrier cuff and a second barrier cuff laterally separated from the first barrier cuff; a proximal bond region connecting the first barrier cuff with the chassis adjacent the first longitudinal side edge, the proximal bond region extending longitudinally for substantially the same length CL as the chassis, the first barrier cuff extending laterally inward toward the second barrier cuff from the proximal bond region to a distal edge; a first end bond region adjacent the first lateral end edge of the chassis, the first end bond region connecting the first barrier cuff with the chassis, wherein the first end bond region extends longitudinally inward from the first lateral end edge and extends laterally inward from the proximal bond region toward the distal edge to define a first void region where the first barrier cuff is not bonded with the chassis between the first end bond region and the proximal bond region, wherein the first void region defines a first void volume adapted to accept bodily exudates; a second end bond region adjacent the second lateral end edge of the chassis, the second end bond region connecting the first barrier cuff with the chassis, wherein second end bond region extends longitudinally inward from the second lateral end edge and extends laterally inward from the proximal bond region toward the distal edge to define a second void region where the first barrier cuff is not bonded with the topsheet between the second end bond region and the proximal bond region, wherein the second void region defines a second void volume adapted to accept bodily exudates; and wherein the first barrier cuff comprises a central segment extending longitudinally between a first segment and a second segment, the first segment extending longitudinally between the first end bond region and the central segment, and the second segment extending longitudinally between the second end bond region and the central segment, wherein the first barrier cuff is not bonded with the chassis between the proximal bond region and the distal edge in the first segment, the central segment, and the second segment, and wherein the first segment of the first barrier cuff comprises: a first cuff height defined by a distance between the proximal bond region and the distal edge, the second segment comprises a second cuff height defined by a distance between the proximal bond region and the distal edge, and the central segment comprises a central cuff height defined by a distance between the proximal bond region and the distal edge, and wherein the central cuff height is less than the first height and the second cuff height; and wherein the first cuff height and the second cuff height of the first absorbent article are larger than the first cuff height and the second cuff height of the second absorbent article. In some forms, the first gender is a male gender and the second gender is a female gender.

### BRIEF DESCRIPTION OF THE DRAWINGS

FIG. 1A is a plan view of an absorbent article in the form of a taped diaper that may include leg gasketing systems in accordance with the present disclosure with the portion of the diaper that faces away from a wearer oriented towards the viewer.

FIG. 1B is a plan view of the absorbent article of FIG. 1A that may include leg gasketing systems in accordance with the present disclosure with the portion of the diaper that faces toward a wearer oriented towards the viewer.

FIG. 2A is a front perspective view of an absorbent article that may include leg gasketing systems in accordance with the present disclosure.

FIG. 2B is a front view of the absorbent article of FIG. 2A.

FIG. 2C is a rear view of the absorbent article of FIG. 2A.

FIG. 3 is a plan view of a chassis with a leg gasketing system for use on a taped diaper or a pant diaper with a portion of the chassis that faces toward a wearer oriented towards the viewer.

FIG. 3A a plan view of a chassis with a leg gasketing system for use on a taped diaper or a pant diaper with a portion of the chassis that faces toward a wearer oriented towards the viewer illustrating inwardly and outwardly positioned cuff elastic strands.

FIG. 3B is a detailed view of an example end bond region.

FIG. 4 is a cross-sectional view of the chassis and leg gasketing system of FIG. 3 taken along line 4-4.

FIG. 4A is a cross-sectional view of the chassis and leg gasketing system of FIG. 3 taken along line 4-4 illustrating other configurations of the barrier cuffs and leg cuffs.

FIG. 5A is a cross-sectional view of the chassis and leg gasketing system of FIG. 3 taken along line 5A-5A.

FIG. 5AA is a cross-sectional view of the chassis and leg gasketing system of FIG. 3 taken along line 5A-5A illustrating other configurations of the barrier cuffs and leg cuffs.

FIG. 5B is a cross-sectional view of the chassis and leg gasketing system of FIG. 3 taken along line 5B-5B.

FIG. 5BA is a cross-sectional view of the chassis and leg gasketing system of FIG. 3 taken along line 5B-5B illustrating other configurations of the barrier cuffs and leg cuffs.

FIG. 6A is a cross-sectional view of the chassis and leg gasketing system of FIG. 3 taken along line 6A-6A.

FIG. 6AA is a cross-sectional view of the chassis and leg gasketing system of FIG. 3 taken along line 6A-6A illustrating other configurations of the barrier cuffs and leg cuffs.

FIG. 6B is a cross-sectional view of the chassis and leg gasketing system of FIG. 3 taken along line 6B-6B.

FIG. 6BA is a cross-sectional view of the chassis and leg gasketing system of FIG. 3 taken along line 6B-6B illustrating other configurations of the barrier cuffs and leg cuffs.

FIGS. 7 and 8 are illustrations of a test apparatus for a Cuff Leakage Test Method.

### DETAILED DESCRIPTION

The following term explanations may be useful in understanding the present disclosure:

The following term explanations may be useful in understanding the present disclosure:

“Absorbent article” is used herein to refer to consumer products whose primary function is to absorb and retain soils and wastes. Absorbent articles can comprise sanitary napkins, tampons, panty liners, interlabial devices, wound dressings, wipes, disposable diapers including taped diapers and diaper pants, inserts for diapers with a reusable outer cover, adult incontinent diapers, adult incontinent pads, and adult incontinent pants. The term “disposable” is used herein to describe absorbent articles which generally are not intended to be laundered or otherwise restored or reused as an absorbent article (e.g., they are intended to be discarded after a single use and may also be configured to be recycled, composted or otherwise disposed of in an environmentally compatible manner).

The term “taped diaper” (also referred to as “open diaper”) refers to disposable absorbent articles having an initial front waist region and an initial back waist region that are not fastened, pre-fastened, or connected to each other as packaged, prior to being applied to the wearer. A taped diaper may be folded about the lateral centerline with the interior of one waist region in surface to surface contact with the interior of the opposing waist region without fastening or joining the waist regions together. Example taped diapers are disclosed in various suitable configurations U.S. Pat. Nos. 5,167,897, 5,360,420, 5,599,335, 5,643,588, 5,674,216, 5,702,551, 5,968,025, 6,107,537, 6,118,041, 6,153,209, 6,410,129, 6,426,444, 6,586,652, 6,627,787, 6,617,016, 6,825,393, and 6,861,571; and U.S. Patent Publication Nos. 2013/0072887 A1; 2013/0211356 A1; and 2013/0306226 A1, all of which are incorporated by reference herein.

The term “pant” (also referred to as “training pant”, “pre-closed diaper”, “diaper pant”, “pant diaper”, and “pull-on diaper”) refers herein to disposable absorbent articles having a continuous perimeter waist opening and continuous perimeter leg openings designed for infant or adult wearers. A pant can be configured with a continuous or closed waist opening and at least one continuous, closed, leg opening prior to the article being applied to the wearer. A pant can be preformed or pre-fastened by various techniques including, but not limited to, joining together portions of the article using any refastenable and/or permanent closure member (e.g., seams, heat bonds, pressure welds, adhesives, cohesive bonds, mechanical fasteners, etc.). A pant can be preformed anywhere along the circumference of the article in the waist region (e.g., side fastened or seamed, front waist fastened or seamed, rear waist fastened or seamed). Example diaper pants in various configurations are disclosed in U.S. Pat. Nos. 4,940,464; 5,092,861; 5,246,433; 5,569,234; 5,897,545; 5,957,908; 6,120,487; 6,120,489; 7,569,039 and U.S. Patent Publication Nos. 2003/0233082 A1; 2005/0107764 A1, 2012/0061016 A1, 2012/0061015 A1; 2013/0255861 A1; 2013/0255862 A1; 2013/0255863 A1; 2013/0255864 A1; and 2013/0255865 A1, all of which are incorporated by reference herein.

The term “feminine hygiene articles” refers to disposable absorbent articles used by women for catamenial protection. Such feminine hygiene articles may include sanitary napkins, tampons, interlabial products, incontinence devices, and pantiliners. Non-limiting examples of panty liners and sanitary napkins include those disclosed in U.S. Pat. Nos. 4,324,246; 4,463,045; 4,342,314; 4,556,146; 4,589,876; 4,687,478; 4,950,264; 5,009,653; 5,267,992; and 6,004,893, all of which are incorporated by reference herein.

An “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

The term “substrate” is used herein to describe a material which is primarily two-dimensional (i.e., in an XY plane) and whose thickness (in a Z direction) is relatively small (i.e., 1/10 or less) in comparison to its length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

The term “nonwoven” refers herein to a material made from continuous (long) filaments (fibers) and/or discontinuous (short) filaments (fibers) by processes such as spunbonding, meltblowing, carding, and the like. In some configurations, a nonwoven may comprise a polyolefin based nonwoven, including but not limited to nonwovens having polypropylene fibers and/or polyethylene fibers and/or bicomponent fibers comprising a polyolefin. Nonlimiting examples of suitable fibers include spunbond, spunlaid, meltblown, spunmelt, solvent-spun, electrospun, carded, film fibrillated, melt-film fibrillated, air-laid, dry-laid, wet-laid staple fibers, and other nonwoven web materials formed in part or in whole of polymer fibers as known in the art, and workable combinations thereof. Nonwovens do not have a woven or knitted filament pattern.

The term “machine direction” (MD) is used herein to refer to the direction of material flow through a process. In addition, relative placement and movement of material can be described as flowing in the machine direction through a process from upstream in the process to downstream in the process.

The term “cross direction” (CD) is used herein to refer to a direction that is generally perpendicular to the machine direction.

Aspects of the present disclosure relate to absorbent articles including a leg gasketing system with features adapted for mitigation of leakage and wearer entanglement while donning. As discussed in more detail below, an absorbent article may comprise: a first waist region and a second waist region longitudinally separated from the first waist region by a crotch region. The absorbent article may further comprise a chassis comprising a topsheet, a backsheet; and an absorbent core positioned between the topsheet and the backsheet. The chassis further comprises a first longitudinal side edge laterally separated from a second longitudinal side edge. The chassis also comprises a first lateral end edge in the first waist region longitudinally separated from a second lateral end edge in second waist region and comprising a longitudinal length CL extending between the first lateral end edge and the second lateral end edge. In some configurations, the longitudinal length CL of the chassis may be from about 400 mm to about 600 mm. The absorbent article also comprises a leg gasketing system comprising a first barrier cuff and a second barrier cuff laterally separated from the first barrier cuff. As discussed in more detail below, the first barrier cuff and/or the second barrier cuff may be bonded with the chassis in proximal bond regions configured to allow for increased cuff heights while at the same time helping to mitigate wearer entanglement during donning. In addition, the first barrier cuff and/or the second barrier cuff may be bonded with the chassis in end bond regions configured to provide void regions between the barrier cuffs and the chassis in waist end regions to help prevent urine from leaking outside the article while the urine is being absorbed by the absorbent core. In some configurations, the leg gasketing system may also comprise leg cuffs bonded with a garment facing surface of the backsheet. Positioning leg cuffs on the garment facing surface of the backsheet also helps provide for increased flexibility with respect to laterally outboard positioning of proximal bond regions to allow barrier cuffs to comprise relatively large cuff heights while at the same time maintaining lateral spacing between the barrier cuffs to help avoid excessive coverage of the absorbent core by barrier cuffs.

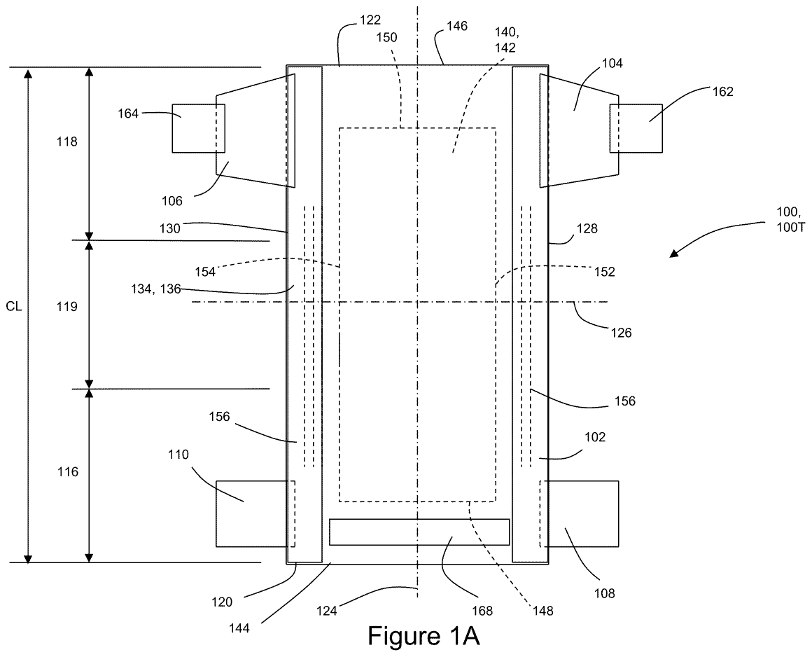

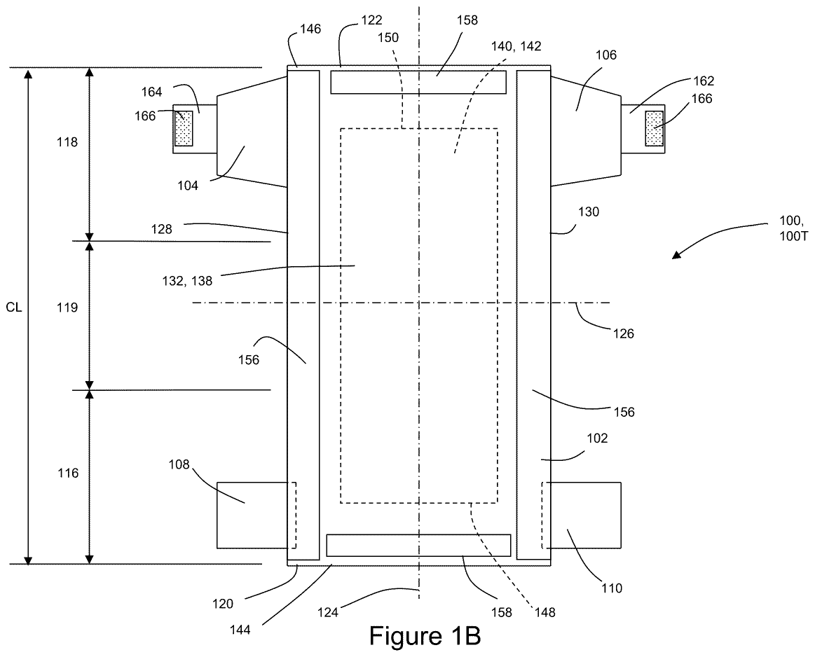

It is to be appreciated that the leg gasketing systems disclosed herein are applicable to work with various types of absorbent articles, such as for example, absorbent articles configured as pant diapers and taped diapers. For the purposes of a specific illustration, FIGS. 1A and 1B show an example of an absorbent article 100 that may be assembled with the leg gasketing systems disclosed herein. In particular, FIG. 1A shows one example of a plan view of an absorbent article 100 configured as a taped diaper 100T, with the portion of the diaper that faces away from a wearer oriented towards the viewer. And FIG. 1B shows a plan view of the diaper 100 with the portion of the diaper that faces toward a wearer oriented towards the viewer. The taped diaper 100T shown in FIGS. 1A and 1B includes an absorbent chassis 102, first and second rear side panels 104 and 106; and first and second front side panels 108 and 110.

As shown in FIGS. 1A and 1B, the diaper 100 and the chassis 102 each include a first waist region 116, a second waist region 118, and a crotch region 119 disposed intermediate the first and second waist regions. The first waist region 116 may be configured as a front waist region, and the second waist region 118 may be configured as back waist region. In some configurations, the length of each of the front waist region, back waist region, and crotch region may be ⅓ of the length of the absorbent article 100.

The diaper 100 may also include a laterally extending first lateral end edge 120 in the first waist region 116 and a longitudinally opposing and laterally extending second lateral end edge 122 in the second waist region 118. To provide a frame of reference for the present discussion, the diaper 100T in FIGS. 1A and 1B is shown with a longitudinal axis 124 and a lateral axis 126. The longitudinal axis 124 may extend through a midpoint of the first end edge 120 and through a midpoint of the second end edge 122. And the lateral axis 126 may extend through a midpoint of a first longitudinal side edge 128 and through a midpoint of a second longitudinal side edge 130 of the chassis 102.

As shown in FIGS. 1A and 1B, the diaper 100 and chassis 102 each include an inner, body facing surface 132, and an outer, garment facing surface 134. And the chassis 102 may include a backsheet 136 and a topsheet 138. The chassis 102 may also include an absorbent assembly 140, including an absorbent core 142, disposed between a portion of the topsheet 138 and the backsheet 136. As discussed in more detail below, the diaper 100 may also include other features, such a leg gasketing system, an elastic waist region, and/or flaps, e.g., side panels and/or ears, to enhance the fits around the legs and waist of the wearer, to enhance the fit around the legs of the wearer.

As shown in FIGS. 1A and 1B, the periphery of the chassis 102 may be defined by a first longitudinal side edge 128, a second longitudinal side edge 130, a laterally extending first end edge 144 disposed in the first waist region 116, and a laterally extending second end edge 146 disposed in the second waist region 118. Both side edges 128 and 130 extend longitudinally between the first end edge 144 and the second end edge 146. As shown in FIG. 1A, the laterally extending end edges 144 and 146 may form a portion of the laterally extending first waist edge 120 in the first waist region 116 and a portion of the longitudinally opposing and laterally extending second waist edge 122 in the second waist region 118. The distance between the first lateral end edge 144 and the second lateral end edge 146 may define a longitudinal length, CL, of the chassis 102. In some configurations, the longitudinal length CL of the chassis 102 may be from about 400 mm to about 600 mm, specifically reciting all 1 mm increments within the above-recited ranges and all ranges formed therein or thereby. In some configurations, the longitudinal length CL of the chassis may be less than about 400 mm.

When the diaper 100 is worn on the lower torso of a wearer, the first waist edge 120 and the second waist edge 122 may encircle a portion of the waist of the wearer. At the same time, the side edges 128 and 130 may encircle at least a portion of the legs of the wearer. And the crotch region 119 may be generally positioned between the legs of the wearer with the absorbent core 142 extending from the front waist region 116 through the crotch region 119 to the back waist region 118.

It is to also be appreciated that a portion or the whole of the diaper 100 may also be made laterally extensible. The additional extensibility may help allow the diaper 100 to conform to the body of a wearer during movement by the wearer. The additional extensibility may also help, for example, the user of the diaper 100, including a chassis 102 having a particular size before extension, to extend the first waist region 116, the second waist region 118, or both waist regions of the diaper 100 and/or chassis 102 to provide additional body coverage for wearers of differing size, i.e., to tailor the diaper to an individual wearer. Such extension of the waist region or regions may give the absorbent article a generally hourglass shape, so long as the crotch region is extended to a relatively lesser degree than the waist region or regions, and may impart a tailored appearance to the article when it is worn.

As previously mentioned, the chassis 102 may include a backsheet 136. The backsheet 136 may also define a portion of the outer, garment facing surface 134 of the chassis 102. The backsheet 136 is generally that portion of the absorbent article 100 positioned proximate to the garment-facing surface of the absorbent assembly 140. The backsheet 136 may be joined to portions of the topsheet 138, the absorbent assembly 140, and/or any other layers of the absorbent article by any attachment methods known to those of skill in the art. The backsheet 136 prevents, or at least inhibits, the bodily exudates absorbed and contained in the absorbent core 142 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials, such as films, which permit vapors to escape from the absorbent article, while still preventing, or at least inhibiting, bodily exudates from passing through the backsheet. The backsheet 136 may also comprise a woven or nonwoven material, polymeric films such as thermoplastic films of polyethylene or polypropylene, and/or a multi-layer or composite materials comprising a film and a nonwoven material, such as having an inner film layer and an outer nonwoven layer or outer cover material. The outer cover material (sometimes referred to as a backsheet nonwoven) may comprise one or more nonwoven materials joined to a film. The outer cover material may form at least a portion of the garment facing surface 134 of the backsheet 136 so that film is not present on the garment facing surface 134. The outer cover material may comprise a bond pattern, apertures, and/or three-dimensional features. The outer cover material may be a hydroentangled nonwoven material or a variable basis weight nonwoven material. Such a material may have one or more layers.

Also described above, the diaper 100 may include a topsheet 138. The topsheet 138 may also define all or part of the inner, wearer facing surface 132 of the chassis 102. The topsheet 138 may be compliant, soft feeling, and non-irritating to the wearer's skin. It may be elastically stretchable in one or two directions. Further, the topsheet 138 may be liquid pervious, permitting liquids (e.g., menses, urine, and/or runny feces) to penetrate through its thickness. A topsheet 138 may be manufactured from a wide range of materials such as woven and nonwoven materials; apertured or hydroformed thermoplastic films; apertured nonwovens, porous foams; reticulated foams; reticulated thermoplastic films; and thermoplastic scrims. Woven and nonwoven materials may comprise natural fibers such as wood or cotton fibers; synthetic fibers such as polyester, polypropylene, or polyethylene fibers; or combinations thereof. If the topsheet 138 includes fibers, the fibers may be spunbond, carded, wet-laid, meltblown, hydroentangled, or otherwise processed as is known in the art.

Topsheets 138 may be selected from high loft nonwoven topsheets, apertured film topsheets and apertured nonwoven topsheets. Apertured film topsheets may be pervious to bodily exudates, yet substantially non-absorbent, and have a reduced tendency to allow fluids to pass back through and rewet the wearer's skin. Exemplary apertured films may include those described in U.S. Pat. Nos. 5,628,097; 5,916,661; 6,545,197; and 6,107,539, all of which are incorporated by reference herein.

As mentioned above, the diaper 100 may also include an absorbent assembly 140 that is joined to the chassis 102. As shown in FIGS. 1A and 1B, the absorbent assembly 140 may have a laterally extending front edge 148 in the front waist region 116 and may have a longitudinally opposing and laterally extending back edge 150 in the back waist region 118. The absorbent assembly may have a longitudinally extending right side edge 152 and may have a laterally opposing and longitudinally extending left side edge 154, both absorbent assembly side edges 152 and 154 may extend longitudinally between the front edge 148 and the back edge 150. The absorbent assembly 140 may additionally include one or more absorbent cores 142 or absorbent core layers. The absorbent core 142 may be at least partially disposed between the topsheet 138 and the backsheet 136 and may be formed in various sizes and shapes that are compatible with the diaper. Exemplary absorbent structures for use as the absorbent core of the present disclosure are described in U.S. Pat. Nos. 4,610,678; 4,673,402; 4,888,231; and 4,834,735, all of which are incorporated by reference herein.

Some absorbent core embodiments may comprise fluid storage cores that contain reduced amounts of cellulosic airfelt material. For instance, such cores may comprise less than about 40%, 30%, 20%, 10%, 5%, or even 1% of cellulosic airfelt material. Such a core may comprise primarily absorbent gelling material in amounts of at least about 60%, 70%, 80%, 85%, 90%, 95%, or even about 100%, where the remainder of the core comprises a microfiber glue (if applicable). Such cores, microfiber glues, and absorbent gelling materials are described in U.S. Pat. Nos. 5,599,335; 5,562,646; 5,669,894; and 6,790,798 as well as U.S. Patent Publication Nos. 2004/0158212 A1 and 2004/0097895 A1, all of which are incorporated by reference herein.

In some configurations, the absorbent assembly 140 may include an acquisition system disposed between the topsheet 138 and a wearer facing side of the absorbent core 142. The acquisition system may be in direct contact with the absorbent core 142 and may comprise a single layer or multiple layers, such as an upper acquisition layer (also referred to herein as a first acquisition layer) facing towards the wearer's skin and a lower acquisition layer (also referred to herein as a second acquisition layer) facing the garment of the wearer. In some embodiments, the acquisition system may function to receive a surge of liquid, such as a gush of urine. As such, the acquisition system may serve as a temporary reservoir for liquid until the absorbent core 142 can absorb the liquid. Exemplary acquisition systems and associated manufacturing processes are described in U.S. Pat. Nos. 8,603,277 and 8,568,566; U.S. Patent Publication Nos. 2012/0316046 A1 and 2014/0163504 A1, all of which are hereby incorporated by reference herein.

As previously mentioned, the diaper 100 may also include a leg gasketing system 156 and an elasticized waistband 158. The leg gasketing system 156 is described in more detail below. The elasticized waistband 158 may provide improved fit and containment and may be a portion or zone of the diaper 100 that may elastically expand and contract to dynamically fit a wearer's waist. The elasticized waistband 158 may extend longitudinally inwardly from the waist edges 120, 122 of the diaper toward the lateral edges 148, 150 of the absorbent core 142. The diaper 100 may also include more than one elasticized waistband 158, for example, having one waistband 158 positioned in the back waist region 118 and one waistband 158 positioned in the front wait region 116, although other configurations may be constructed with a single elasticized waistband 158. The elasticized waistband 158 may be constructed in a number of different configurations including those described in U.S. Pat. Nos. 4,515,595 and 5,151,092; and U.S. Patent Publication Nos. 2020/0375807 A1; 2020/0375815 A1; 2021/0128366 A1; and 2021/0128369 A1, all of which are incorporated by reference. In some configurations, the elasticized waistbands 158 may include materials that have been “prestrained” or “mechanically prestrained” (subjected to some degree of localized pattern mechanical stretching to permanently elongate the material). The materials may be prestrained using deep embossing techniques as are known in the art. In some embodiments, the materials may be prestrained by directing the material through an incremental mechanical stretching system as described in U.S. Pat. No. 5,330,458. The materials are then allowed to return to their substantially untensioned condition, thus forming a zero strain stretch material that is extensible, at least up to the point of initial stretching. Examples of zero strain materials are disclosed in U.S. Pat. Nos. 2,075,189; 3,025,199; 4,107,364; 4,209,563; 4,834,741; and 5,151,092, all of which are incorporated by reference herein.

Taped diapers 100T may be manufactured and provided to consumers in a configuration wherein the front waist region and the back waist region are not fastened, pre-fastened, or connected to each other as packaged, prior to being applied to the wearer. For example, the taped diaper 100 may be folded about a lateral centerline with the wearer facing surface 132 of the first waist region 116 in surface to surface contact with the wearer facing surface 132 of the second waist region 118 without fastening or joining the waist regions together. The rear side panels 104 and 106 and/or the front side panels 108 and 110 may also be folded laterally inward toward the inner, wearer facing surfaces 132 of the waist regions 116 and 118.

The diaper 100T may also include various configurations of fastening elements to enable fastening of the front waist region 116 and the back waist region 118 together to form a closed waist circumference and leg openings once the diaper is positioned on a wearer. For example, as shown in FIGS. 1A and 1B, the diaper 100 may include first and second fastening members 162, 164, also referred to as tabs, connected with the first and second rear side panels 104, 106, respectively. The diaper may also include first and second front side panels 108, 110, that may or may not include fastening members.

With continued reference to FIGS. 1A and 1B, each side panel 104, 106 and/or fastening member 162 and 164 may form a portion of or may be permanently bonded, adhered or otherwise joined directly or indirectly to the chassis 102 laterally inward from the side edge 128 and 130, in one of the front waist region 116 or the back waist region 118. Alternatively, the fastening members 162, 164 may form a portion of or may be permanently bonded, adhered or otherwise joined directly or indirectly to the first and second rear panels 104, 106 at or adjacent the distal edge of the panel and/or the first and second front side panels 108 and 110 at or adjacent the distal edge of the side panel. It is to be appreciated that the fastening members and/or side panels may be assembled in various ways, such as disclosed for example, in U.S. Pat. No. 7,371,302, which is incorporated by reference herein. The fastening members 162, 164 and/or side panels 104, 106, 108, 110 may also be permanently bonded or joined at or adjacent the side edges 128 and 130 of the chassis 102 in various ways, such as for example, by adhesive bonds, sonic bonds, pressure bonds, thermal bonds or combinations thereof, such as disclosed for example, U.S. Pat. No. 5,702,551, which is incorporated by reference herein.

Referring now to FIG. 1B, the first fastening member 162 and/or the second fastening member 164 may include various types of releasably engageable fasteners. The first and second fastening members 162 and/or 164 may also include various types of refastenable fastening structures. For example, the first and second fastening members 162 and 164 may include mechanical fasteners, 166, in the form of hook and loop fasteners, hook and hook fasteners, macrofasteners, buttons, snaps, tab and slot fasteners, tape fasteners, adhesive fasteners, cohesive fasteners, magnetic fasteners, hermaphroditic fasteners, and the like. Some examples of fastening systems and/or fastening members 162, 164 are discussed in U.S. Pat. Nos. 3,848,594; 4,662,875; 4,846,815; 4,894,060; 4,946,527; 5,151,092; 5,221,274; 6,251,097; 6,669,618; 6,432,098; and U.S. Patent Publication Nos. 2007/0078427 A1 and 2007/0093769 A1, all of which are incorporated by reference herein.

As previously mentioned, the fastening members 162 and 164 may be constructed from various materials and may be constructed as a laminate structure. The fastening members 162 and 164 may also be adapted to releasably and/or refastenably engage or connect with another portion of the diaper 100. For example, as shown in FIG. 1A, the diaper 100 may include a connection zone 168, sometimes referred to as a landing zone, in the first waist region 116. As such, when the taped diaper 100 is placed on a wearer, the fastening members 162 and 164 may be pulled around the waist of the wearer and connected with the connection zone 168 in the first waist region 116 to form a closed waist circumference and a pair of laterally opposing leg openings. It is to be appreciated that the connection zone may be constructed from a separate substrate that is connected with the chassis 102 of the taped diaper. In some embodiments, the connection zone may be integrally formed as part of the backsheet 136 of the diaper 100 or may be formed as part of the first and second front panels 108, 110, such as described in U.S. Pat. Nos. 5,735,840 and 5,928,212, all of which are incorporated by reference herein.

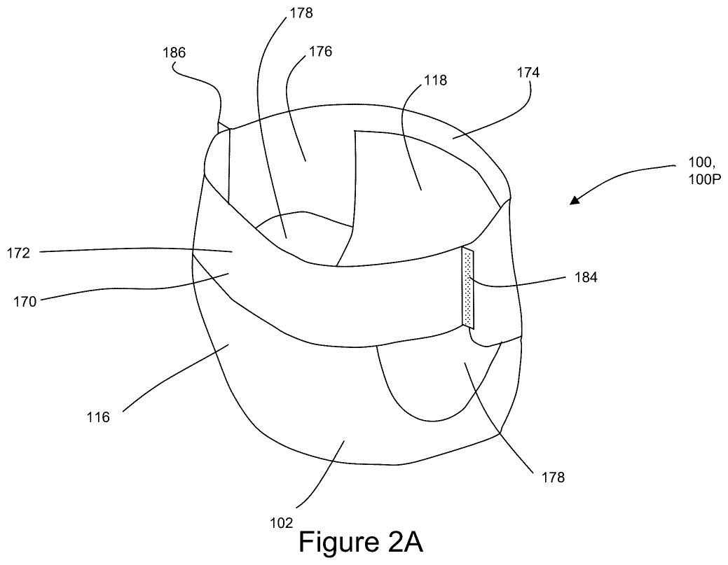

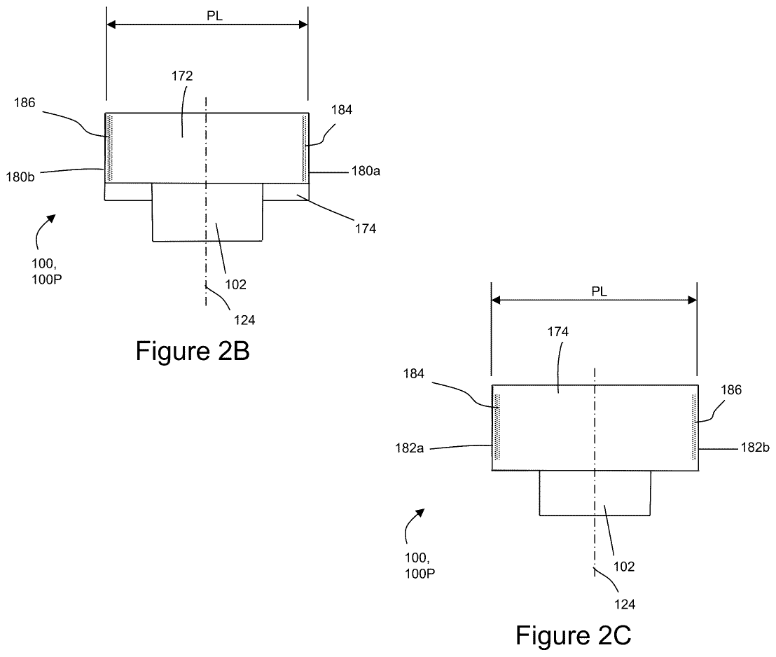

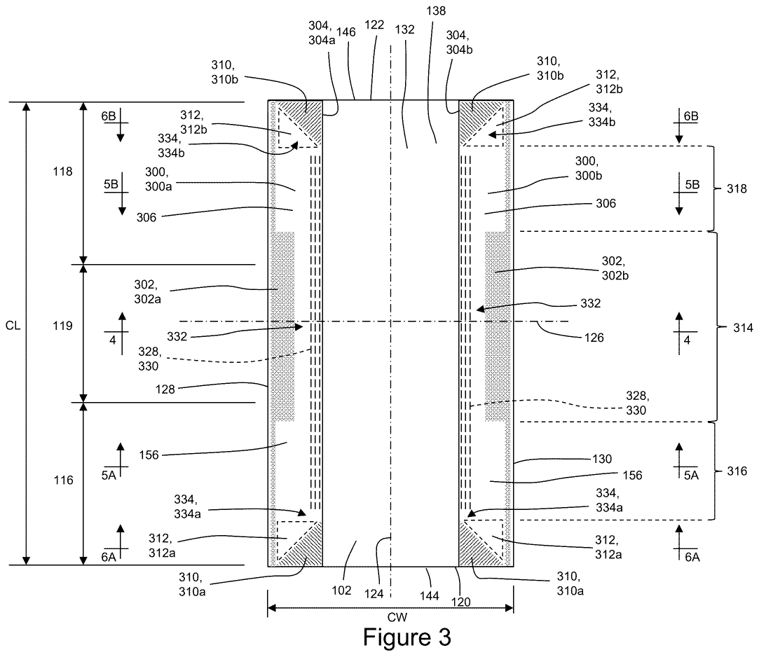

As previously mentioned, absorbent articles 100 may also be configured as diaper pants 100P having a continuous perimeter waist opening and continuous perimeter leg openings. For example, FIG. 2A shows a perspective view of an absorbent article 100 in the form of a diaper pant 100P in a pre-fastened configuration, and FIGS. 2B and 2C show front and rear plan views of the diaper pant 100P. The diaper pant 100P may include a chassis 102, such as discussed above with reference to FIG. 1A, and a ring-like elastic belt 170, such as shown in FIG. 2A. In some configurations, a first elastic belt 172 and a second elastic belt 174 are bonded together to form the ring-like elastic belt 170. As such, diaper pants may be manufactured with the ring-like elastic belt 174 and provided to consumers in a configuration wherein the front waist region 116 and the back waist region 118 of the chassis 102 are connected to each other as packaged, prior to being applied to the wearer. As such, diaper pants may have a continuous perimeter waist opening 176 and continuous perimeter leg openings 178 such as shown in FIG. 2A. Example elastic belt 170 configurations are disclosed in U.S. Patent Publication Nos. 2018/0168878 A1; 2018/0168877 A1; 2018/0168880 A1; 2018/0170027 A1; 2018/0169964 A1; 2018/0168879 A1; 2018/0170026 A1; 2018/0168889 A1; 2018/0168874 A1; 2018/0168875 A1; 2018/0168890 A1; 2018/0168887 A1; 2018/0168892 A1; 2018/0168876 A1; 2018/0168891 A1; 2019/0070042 A1; 2019/0070041 A1; 2021/0282797 A1; 2021/0275362 A1; 2022/0142828 A1; and 2022/0362068 A1, which are all incorporated by reference herein.

As previously mentioned, the ring-like elastic belt 170 may be defined by a first elastic belt 172 connected with a second elastic belt 174. As shown in FIGS. 2A-2C, the first elastic belt 172 extends between a first longitudinal side edge 180a and a second longitudinal side edge 180b. And the second elastic 174 belt extends between a first longitudinal side edge 182a and a second longitudinal side edge 182b. The distance between the first longitudinal side edge 180a and the second longitudinal side edge 180b defines a pitch length, PL, of the first elastic belt 172, and the distance between the first longitudinal side edge 182a and the second longitudinal side edge 182b defines the pitch length, PL, of the second elastic belt 174. The first elastic belt 172 is connected with the first waist region 116 of the chassis 102, and the second elastic belt 108 is connected with the second waist region 116 of the chassis 102. As shown in FIGS. 2A-2C, opposing end regions of the first elastic belt 172 are connected with opposing end regions of the second elastic belt 174 at a first side seam 184 and a second side seam 186 to define the ring-like elastic belt 170 as well as the waist opening 176 and leg openings 178. It is to be appreciated that the ring-like elastic belt may be formed by joining a first elastic belt to a second elastic belt with permanent side seams or with openable and reclosable fastening systems disposed at or adjacent the laterally opposing sides of the belts. It is also to be appreciated that the first elastic belt 172 and the second elastic belt 174 may discrete components that are connected with each other by the chassis 102. In some configurations, the first elastic belt 172 and the second elastic belt 174 may be integrally formed and may be connected with one more outer cover substrates that may extend contiguously from the from the first waist edge 120 to the second waist edge 122.

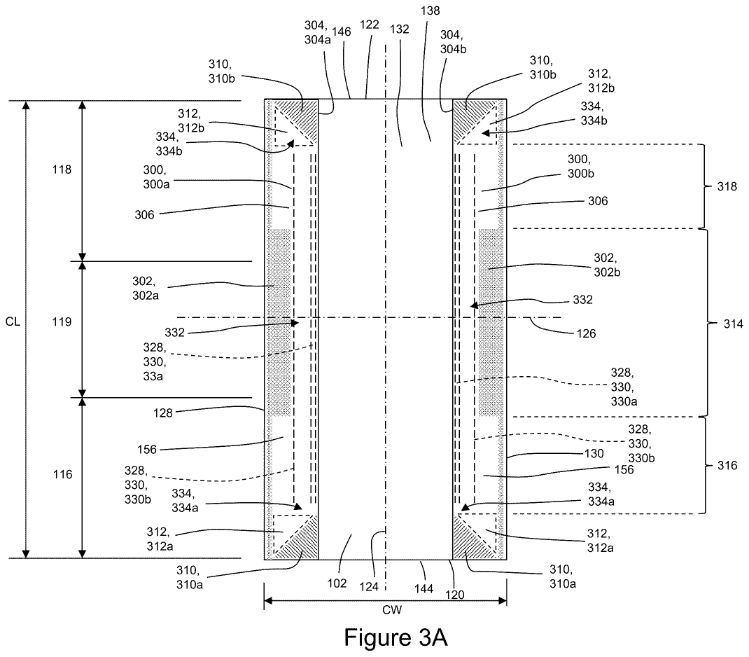

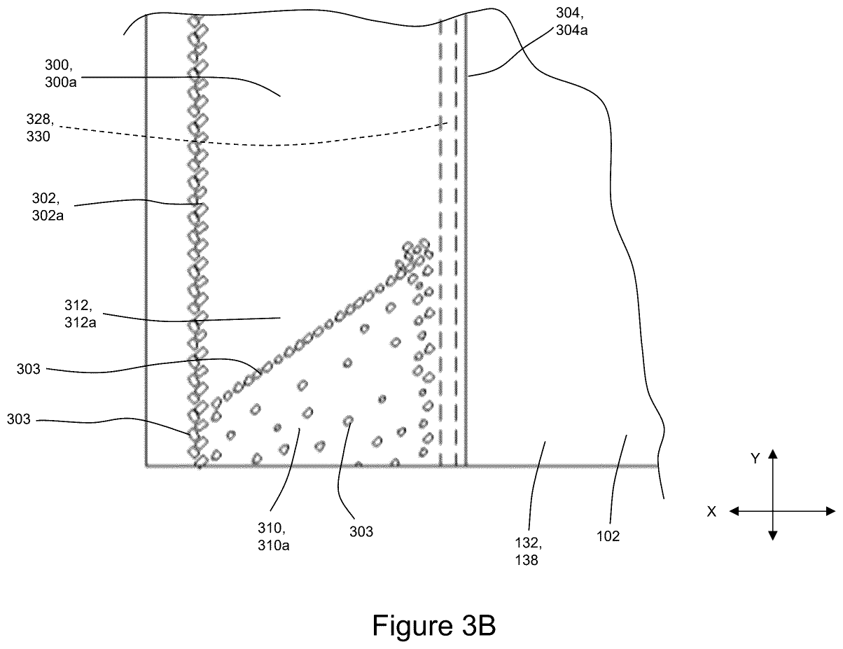

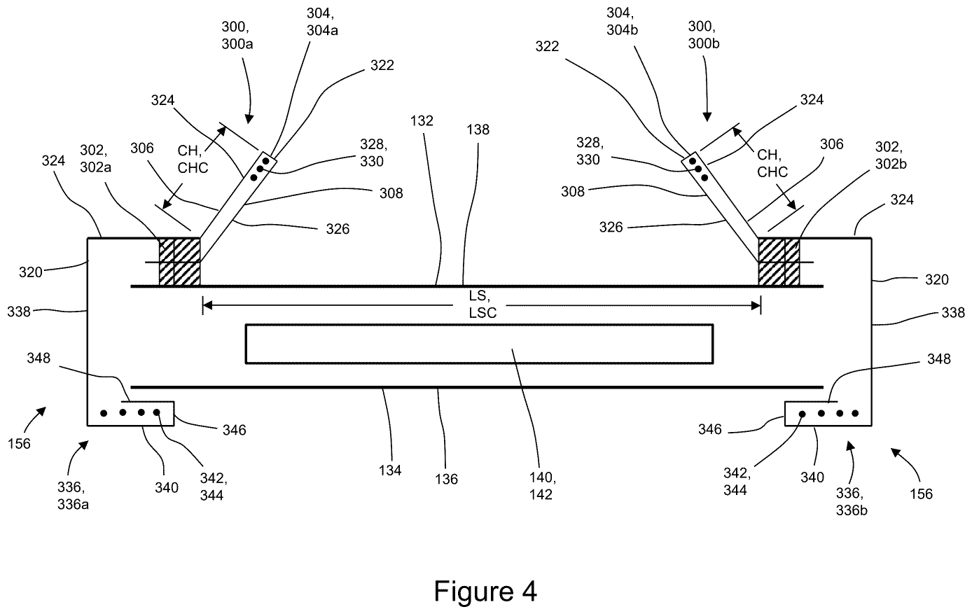

As previously mentioned, the diapers 100 may also include a leg gasketing system 156 that may be configured in various ways to help reduce the leakage of body exudates in the leg regions. For example, FIG. 3 shows a plan view a chassis 102 that comprises a leg gasketing system 156 that may be incorporated into taped diapers 100T and pant diapers 100P, such as the examples disclosed herein. It is also to be appreciated that leg gasketing systems 156 may include various structural components that are sometimes also referred to as leg cuffs, leg bands, side flaps, barrier cuffs, elastic cuffs, and gasketing cuffs. Examples of such structural components and various arrangements thereof may include those described in U.S. Pat. Nos. 3,860,003; 4,695,278; 4,704,115; 4,795,454; 4,909,803; and 8,939,957; U.S. Patent Publication No. 2009/0312730 A1; and PCT Patent Publication Nos. WO2022/183432 A1 and WO2022/183962 A1, all of which are incorporated by reference herein.

With reference to FIGS. 3, 4, and 5A-6B, a leg gasketing system 156 comprising barrier cuffs 300 may be connected with the chassis 102 by proximal bond regions 302. The barrier cuffs 300 may extend laterally inward from the proximal bond regions 302 to distal edges 304. In particular, the leg gasketing system 156 may comprise a first barrier cuff 300a and a second barrier cuff 300b laterally separated from the first barrier cuff 300a. A first proximal bond region 302a connects the first barrier cuff 300a with the chassis 102 adjacent the first longitudinal side edge 128, and a second proximal bond region 302b connects the second barrier cuff 300b with the chassis 102 adjacent the second longitudinal edge 130. In some configurations, the proximal bond regions 302 may connect the barrier cuffs 300 with the topsheet 138 and may extend longitudinally for substantially the same length CL of the chassis 102. In some configurations, the proximal bond regions 302 and/or barrier cuffs 300 may extend longitudinally for the entire length CL or less than the length CL of the chassis. The first barrier cuff 300a may extend laterally inward from the first proximal bond region 302a to a first distal edge 304a, and the second barrier cuff 300b may extend laterally inward from the second proximal bond region 302b to a second distal edge 304b. As such, the first and second barrier cuffs 300a, 300b may extend laterally inward across the wearer facing surface 132 of the chassis toward each other from the first and second proximal bond regions 302a, 302b. The barrier cuffs 300 each include a wearer facing surface 306 and a garment facing surface 308. In turn, the garment facing surface 308 of the barrier cuffs 300 may be positioned in a facing relationship with the wearer facing surface 132 of the topsheet 138. In some configurations, the barrier cuffs 300 may extend laterally inward so as to overlap the wearer facing surface 132 of the absorbent assembly 140. In some configurations, the barrier cuffs 300 may be positioned entirely laterally outside of the absorbent assembly 140.

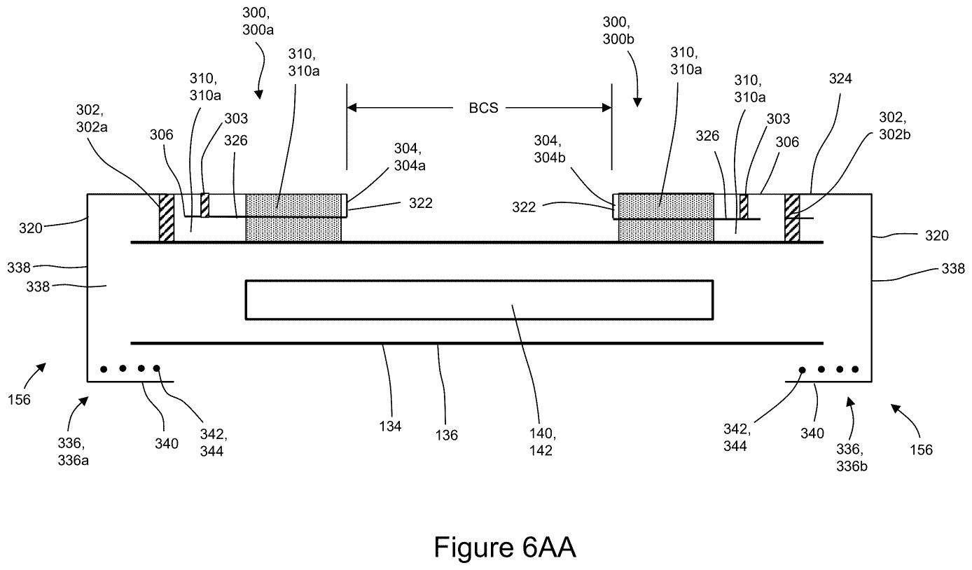

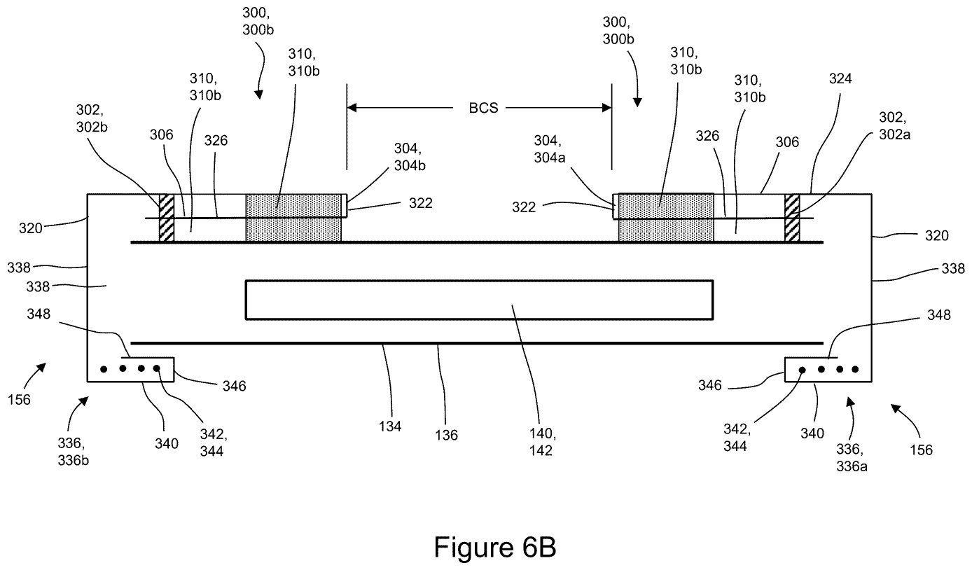

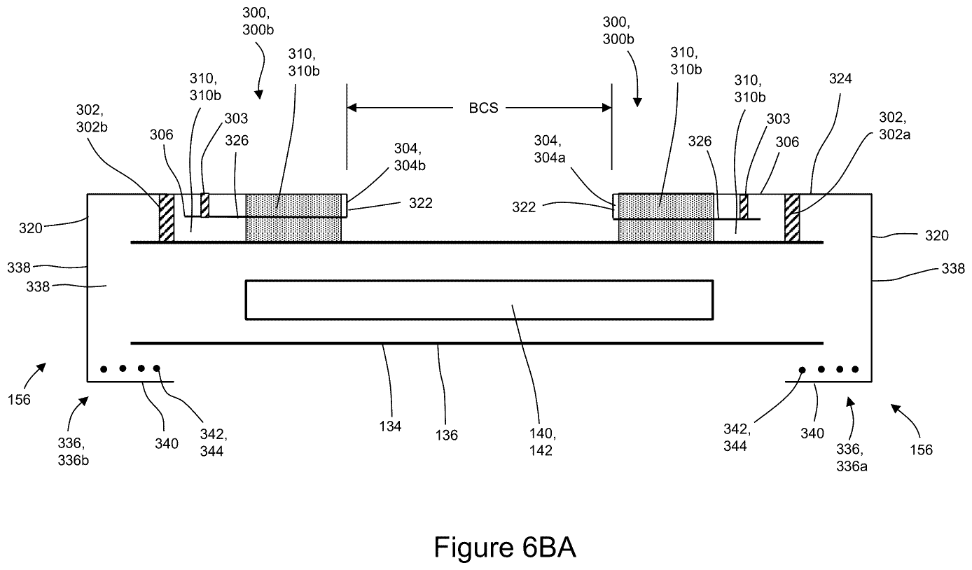

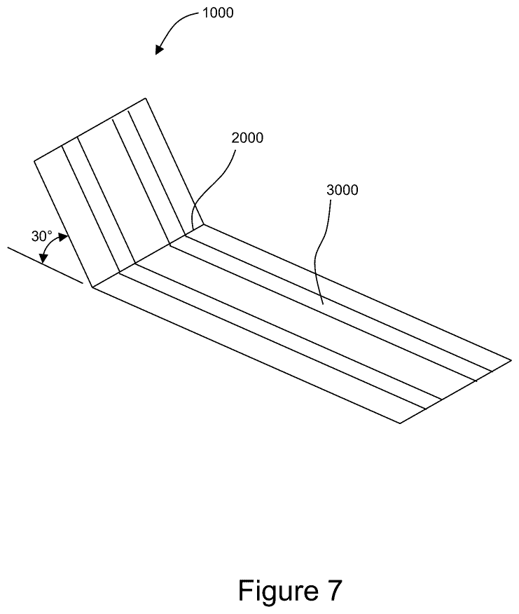

With reference to FIGS. 3 and 6A, end bond regions 310 may also bond the barrier cuffs 300 with the chassis 102 adjacent the first lateral end edge 144 and/or the second lateral end edge 146. In some configurations, first end bond regions 310a may connect the first barrier cuff 300a and the second barrier cuff 300b with the topsheet 138 adjacent the first lateral end edge 144 of the chassis 102 in the first waist region 116. The first end bond regions 310a extend longitudinally inward from the first lateral end edge 144 of the chassis 102 and also extend laterally inward from the proximal bond regions 302 toward the distal edges 304 of the barrier cuffs 300 to define first void regions 312a in the first waist region 116, generically represented by a dashed triangular shape in FIG. 3. Each first void region 312a defines a void volume between the barrier cuff 300 and the topsheet 138 that is adapted to accept bodily exudates in the first waist region 116.

With continued reference to FIGS. 3 and 6B, second end bond regions 312b may connect the first barrier cuff 300a and the second barrier cuff 300b with the topsheet 138 adjacent the second lateral end edge 146 of the chassis in the second waist region 118. The second end bond regions 312 extend longitudinally inward from the second lateral end edge 146 of the chassis 102 and also extend laterally inward from the proximal bond regions 302 toward the distal edges 304 of the barrier cuffs 300 to define second void regions 312b in the second waist region 118, generically represented by a dashed triangular shape in FIG. 3. Each second void region 312b defines a void volume between the barrier cuff 300 and the topsheet 138 that is adapted to accept bodily exudates in the second waist region 118. It is to be appreciated that the end bond regions 310 may be configured to define different shapes and sizes than those shown in FIG. 3, and in turn, may define void regions 312 having different shapes and sizes.

It is to be appreciated that the end bond regions 310 may extend laterally inward to such an extent such that the distal edges 304 of the barrier cuffs 300 are bonded with the chassis 102. In some configurations, the distal edges 304 of the barrier cuffs 300 and some relatively small portions of the barrier cuffs 300 may not be bonded with the chassis 102 by the end bond regions 310.

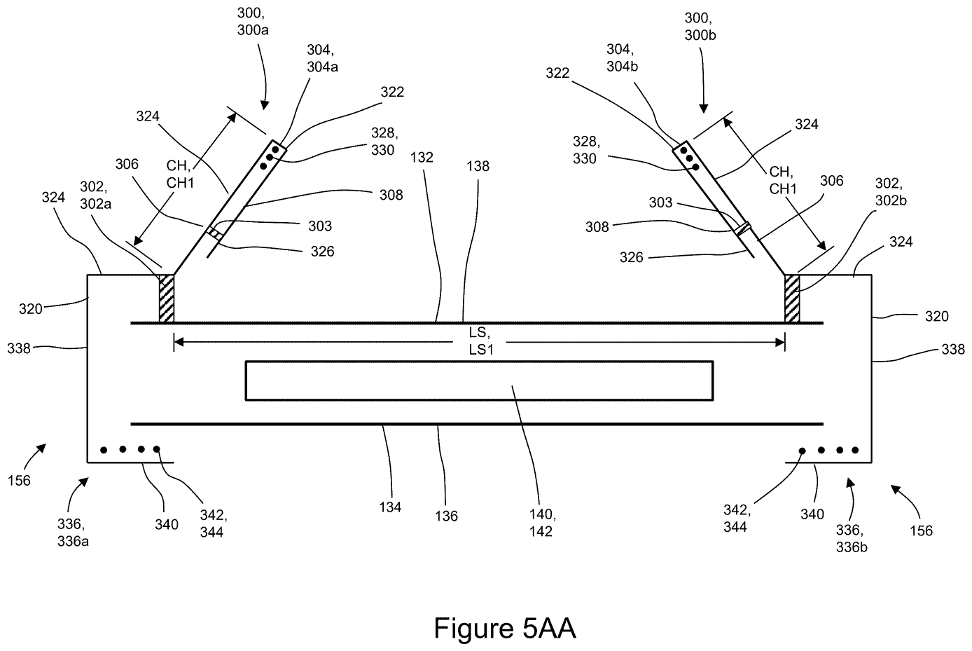

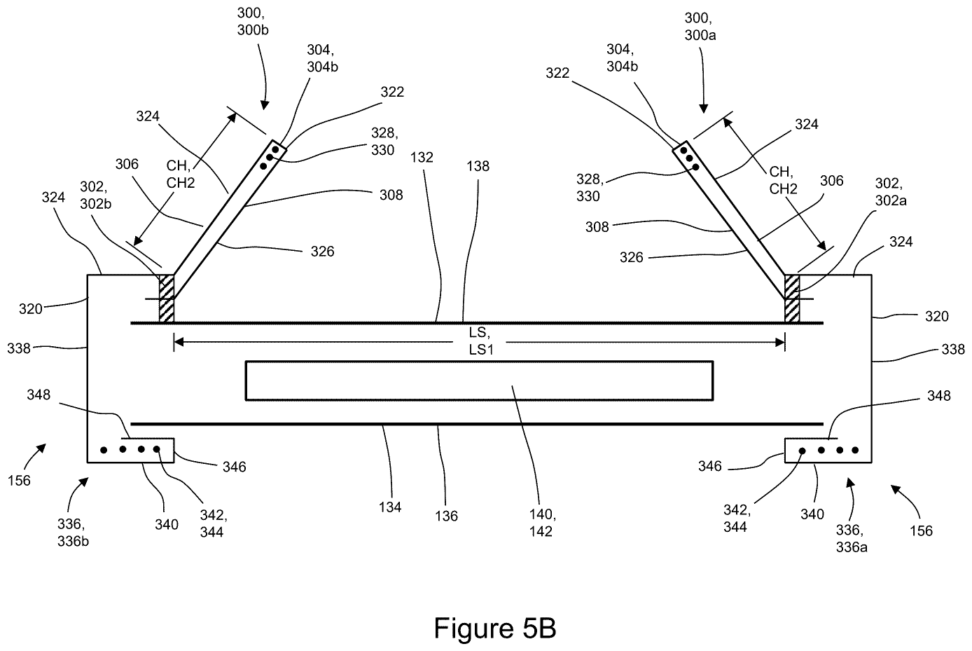

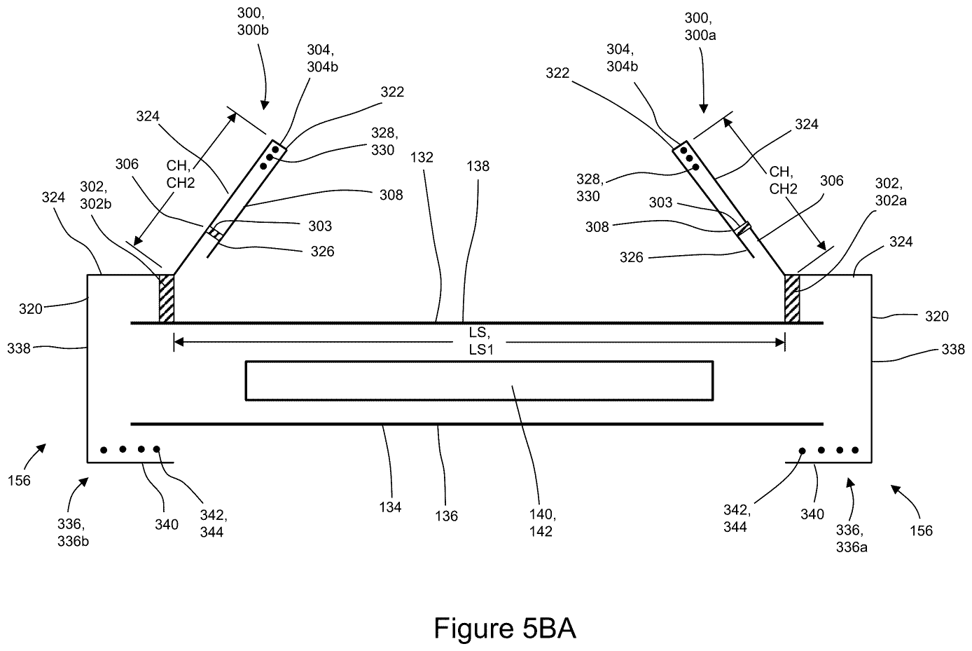

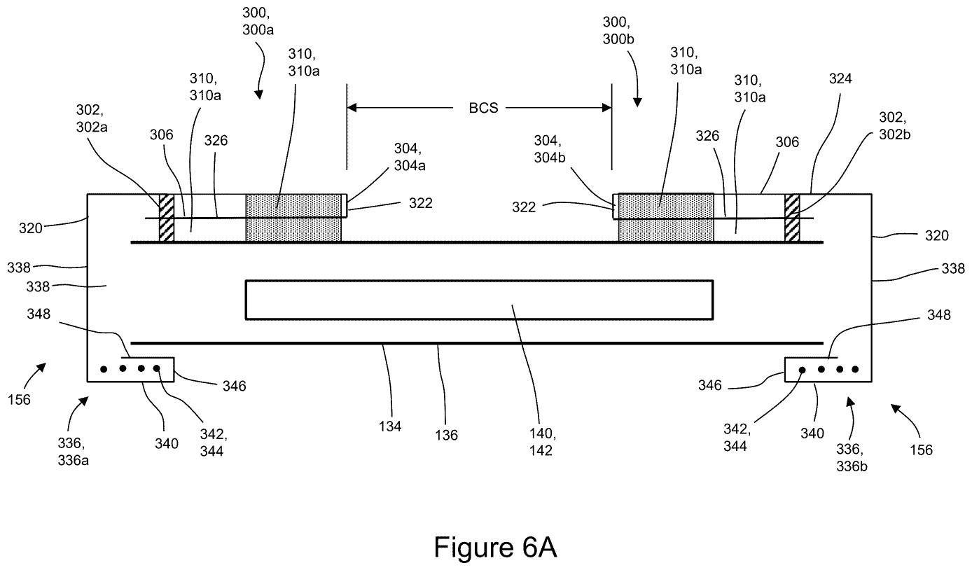

As shown in FIGS. 5A-6B, the barrier cuffs 300 may comprise a cuff height CH defined by a distance between the proximal bond region 302 and the distal edge 304. It is to be appreciated that relatively large cuff heights may help to increase the effectiveness of the barrier cuffs 300 to prevent leakage of bodily exudates from the sides of the chassis 102. In some configurations, the cuff height CH may be from about 40 mm to about 60 mm, specifically reciting all 1 mm increments within the above-recited ranges and all ranges formed therein or thereby. In some configurations, the cuff height CH may be at least about 45 mm. However, in some instances, relatively large cuff heights may cause unintended problems. In some instances, the likelihood of a wearer to become entangled with barrier cuffs 300 having relatively large cuff heights may be increased. For example, a wearer's feet may become entangled with barrier cuffs 300 in the crotch region 119 of a diaper pant 100P when donning a diaper pant configured with relatively large cuff heights. To help mitigate problems associated with wearer entanglement, the barrier cuffs 300 may comprise cuff heights that may vary along the longitudinal length of chassis 102. For example, in some configurations, the barrier cuffs 300 may comprise relatively small cuff heights CH in the crotch region 119 than in the first waist region 116 and/or the second waist region 118. As such, barrier cuffs 300 comprising a relatively small cuff height in the crotch region 119 may help reduce the likelihood of wearer entanglement during donning. In turn, the barrier cuffs 300 may comprise relatively large cuff heights in the first waist region 116 and/or the second waist region 118 to help increase the effectiveness of the barrier cuffs 300 to prevent leakage of bodily exudates from the sides of chassis 102 in the waist regions 116, 118.

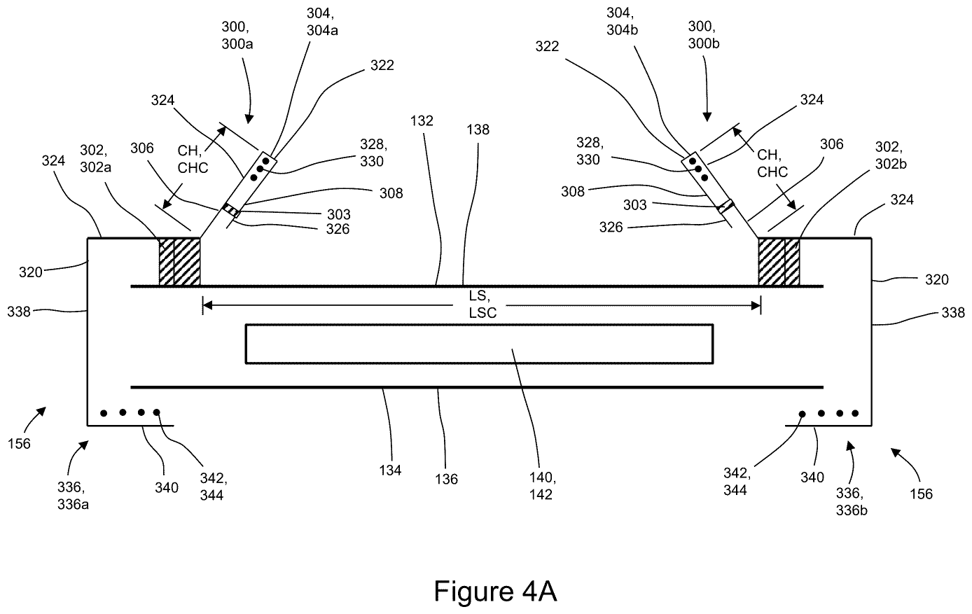

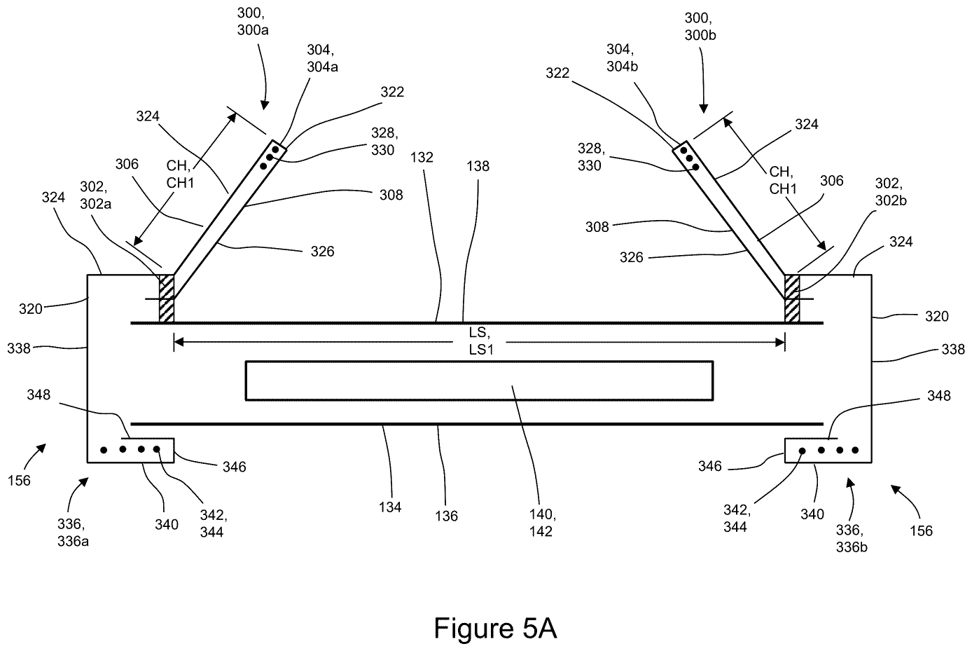

As shown in FIGS. 3, 4, 5A, and 5B, each barrier cuff 300 may comprise a central segment 314 extending longitudinally between a first segment 316 and a second segment 318. The first segment 316 extends longitudinally between the first end bond region 310a and the central segment 314, and the second segment 318 extends longitudinally between the second end bond region 310b and the central segment 314. The barrier cuffs 300 are not bonded with the chassis 102 laterally between the proximal bond region 302 and the distal edge 304 in the first segment 316, the central segment 314, and the second segment 318. The first segment 316 of each barrier cuff 300 may comprise a first cuff height CH1 defined by a distance between the proximal bond region 302 and the distal edge 304. The second segment 318 of each barrier cuff 300 may comprise a second cuff height CH2 defined by a distance between the proximal bond region 302 and the distal edge 304. The central segment 314 of each barrier cuff 300 may comprise a central cuff height CHC defined by a distance between the proximal bond region 302 and the distal edge 304. As shown in FIGS. 4, 5A, and 5B, the proximal bond region 302 may be configured such that the central cuff height CHC is less than the first cuff height CH1 and the second cuff height CH2.

It is to be appreciated that various different structural features may be utilized to vary the cuff heights CH. For example, the proximal bond regions 302 may have relatively wider lateral widths in the central segments 314 than in the first and/or second segments 316, 318. In some configurations, the proximal bond regions 302 may be separated from each other by relatively larger lateral distances in the first and/or second segments 316, 318 than in the central segment 314. For example, as shown in FIGS. 4 and 5A-6B, the first proximal bond region 302a may be laterally spaced from the second proximal bond region 302b by a separation distance LS. The separated distance LS in the central segment 314 may be a central separation distance LSC; the separation distance LS in the first segment 316 may be a first separation distance LS1; and the separation distance in the second segment 318 may be a second separation distance LS2. As such, in some configurations, the central separation distance LSC may be less than the first separation distance LS1 and/or the second separation distance LS2. In some configurations, the central separation distance LSC may be from about 120 mm to about 170 mm, and the first separation distance LS1 and/or the second separation distance LS2 may be from about 130 mm to about 190 mm, specifically reciting all 1 mm increments within the above-recited ranges and all ranges formed therein or thereby.

It is also to be appreciated the cuff heights CH can vary by various different amounts. For example, in some configurations, the first cuff height CH1 and/or the second cuff height CH2 may be from about 40 mm to about 60 mm, specifically reciting all 1 mm increments within the above-recited ranges and all ranges formed therein or thereby. In some configurations, the first cuff height CH1 and/or the second cuff height CH2 may be greater than about 60 mm. In some configurations, the central cuff height CHC may be from about 30 mm to about 40 mm, specifically reciting all 1 mm increments within the above-recited ranges and all ranges formed therein or thereby. In some configurations, central cuff height CHC may be from about 50% to about 90% of the first cuff height CH1 and/or the second cuff height CH2, specifically reciting all 1% increments within the above-recited ranges and all ranges formed therein or thereby.

It is to be appreciated that the absorbent articles 100 and leg gasketing systems 156 may be configured with different features adapted to accommodate or fit different genders of wearers. For example, in some configurations, a first absorbent article may be configured for a wearer of a first gender and a second absorbent article may be configured for a wearer of a second gender. For example, the barrier cuffs 300 of the first absorbent article may comprise cuff heights CH that may be larger than cuff heights CH of the second absorbent article. In some configurations, the first cuff height CH1 of the first absorbent article may be larger than the first cuff height CH1 of the second absorbent article, and/or the second cuff height CH2 of the first absorbent article may be larger than the second cuff height CH2 of the second absorbent article. In some configurations, the first gender may be a male gender and the second gender may be a female gender.

With reference to FIGS. 6A and 6B, the distal edges 304 of the barrier cuffs 300 may be laterally separated from each other by a barrier cuff separation distance BCS, which may be defined as a laterally extending distance between the first distal edge 304a of the first barrier cuff 300a and the second distal edge 304b of the second barrier cuff 300b when the first and second barrier cuffs are laid completely flat on the wearer facing surface 132 of the chassis 102. In some configurations, the barrier cuff separation distance BCS may be at least about 80 mm. In some configurations, the barrier cuff separation distance BCS may be from about 70 mm to about 140 mm, specifically reciting all 1 mm increments within the above-recited ranges and all ranges formed therein or thereby. In some configurations, the barrier cuff separation distance BCS may be from about 60 mm to about 70 mm, specifically reciting all 1 mm increments within the above-recited ranges and all ranges formed therein or thereby.

It is to be appreciated that the barrier cuff separation distance BCS may be decreased with an increased cuff height CH. However, a relatively small the barrier cuff separation distance BCS may increase the likelihood of a wearer to discharge bodily exudates on the wearer facing surface 306 of the barrier cuffs 300 rather than between the garment facing surface 308 of the barrier cuffs 300 and the topsheet 138. As such, in some configurations, the barrier cuffs 300, and in particular, the separation distance LS between the proximal bond regions 302 may be increased to help maintain a desired barrier cuff separation distance BCS when utilizing barrier cuffs with relatively large cuff heights CH. However, as discussed in more detail below, in some configurations, the separation distance LS between the proximal bond regions 302 may be limited by a chassis width CW defined by a lateral distance extending between the first side edge 128 and the second side edge 130 of the chassis 102. In some configurations, the chassis width CW may be from about 180 mm to about 230 mm, specifically reciting all 1 mm increments within the above-recited ranges and all ranges formed therein or thereby.

It is to be appreciated that the proximal bond regions 302 and the end bond regions 310 may be configured in various ways to bond the barrier cuffs 300 with the chassis 102. In some configurations, the proximal bond regions 302 and/or the end bond regions 310 may comprise adhesive that bonds with barrier cuffs 300 with the chassis 102. In some configurations, the proximal bond regions 302 and/or the end bond regions 310 may comprise mechanical bonds that bond with barrier cuffs 300 with the chassis 102. It is to be appreciated that the mechanical bonds may be formed in various ways, as for example, heat, pressure, and/or ultrasonic bonds. In some configurations, the proximal bond regions 302 and/or the end bond regions 310 may comprise combinations of adhesive and mechanical bonds.

In some configurations, the proximal bond regions 302 and/or the end bond regions 310 may comprise a plurality of intermittent bonds. FIG. 3B shows an example of a proximal bond region 302 and an end bond region 310 that comprise a plurality of discrete bonds 303. In some configurations, the end bond region 310 may comprise a relatively constant number of bonds aligned in a lateral direction X throughout a longitudinal length of the end bond region. In some configurations, the end bond region 310 may comprise a variable number of bonds aligned in a longitudinal direction Y throughout a lateral width of the end bond region 310. In some configurations, the discrete bonds 303 may be concentrated in areas where higher bond strengths are desired, such as for example, adjacent the distal edges 304 of the barrier cuffs 300, such as shown in FIG. 3B.

It is to be appreciated that the barrier cuffs 300 may be configured in various ways and may be constructed from one or more cuff substrates. In some configurations, the barrier cuffs 300 may be formed from a plurality of discrete cuff substrates bonded together in various ways. In some configurations, the barrier cuffs 300 may be formed from a single discrete cuff substrate. For example, as shown in FIGS. 4-6B, the first barrier cuff 300a and the second barrier cuff 300b may each comprise a cuff substrate 320 folded along a first fold line 322 to position a first portion 324 of the cuff substrate 320 in a facing relationship with a second portion 326 of the cuff substrate 320. In some configurations, the first fold lines 322 may define the distal edges 304 of the barrier cuffs 300. As shown in FIGS. 4-6B, the first portion 324 of the cuff substrate 320 may extend laterally inward from the proximal bond region 302 to the first fold line 322 and may define the wearer surface 306 of the barrier cuff 300. With continued reference to FIGS. 4-6B, the second portion 326 of the cuff substrate 320 may extend laterally outward from the first fold line 322 to the proximal bond region 302. In some configurations, the second portion 326 of the cuff substrate 320 is bonded between the first portion 324 of the cuff substrate 320 and the chassis 102 in the proximal bond region 302. As such, the second portion 326 of the cuff substrate 320 may define the garment facing surface 308 of the barrier cuff 300. It is to be appreciated that the second portion 326 of the cuff substrate 320 may extend laterally outward from the first fold line 322 for various distances. For example, the second portion 326 of the cuff substrate 320 may not extend laterally outward from the first fold line 322 all the way to the proximal bond region 302. As such, the garment facing surface 308 of the barrier cuff 300 may be defined by both the first portion 324 and the second portion 326 of the cuff substrate 320, such as shown in FIGS. 4A, 5AA, 5BA, 6AA, and 6BA wherein the second portion 326 of the cuff substrate 320 is bonded with the first portion 324 with a hem bond 303.

It is to be appreciated that the cuff substrate 320 may be constructed from various types of materials. For example, the cuff substrate 320 may comprise a substantially liquid impervious material. In some configurations, the cuff substrate 320 may comprise an SMS nonwoven or an SMMS nonwoven material, or a nonwoven component layer comprising fine fibers having an average diameter of less than 1 micron. In some configurations, the cuff substrate 320 may comprise a combination of nonwoven fabric webs including spunbond, meltblown, spunbond (“SMS”) webs comprising outer layers of spunbond thermoplastics (e.g., polyolefins) and an interior layer of meltblown thermoplastics. Example cuff substrates may comprise those of SMS type available from Toray Polytech Nantong China with tradename LIVSEN SMS 13, available from FON Hazlet NJ with tradename SM15009270, and available from Fibertex Aalborg Denmark with tradename B10160HS.

In some configurations, the cuff substrate 320 may be treated, by region or in part of a region, with a lotion or a hydrophobic surface coating to provide various physical properties. In some configurations, the cuff substrate 320 may comprise a hydrostatic head of greater than about 2 mbar, or greater than about 3 mbar, or greater than about 4 mbar, measured according to the Hydrostatic Head Test herein. In some configurations, the cuff substrate 320 may comprise a hydrostatic head of less than about 200 mbar, or less than about 100 mbar, or less than about 75 mbar, or less than about 50 mbar, or less than about 25 mbar, or less than about 15 mbar, measured according to the Hydrostatic Head Test herein.

In some configurations, the barrier cuffs 300 may comprise a cuff elastic material 328 bonded with the cuff substrate 320 under tension. As such, the tension of the cuff elastic material 328 may be configured so as to cause the distal edges 304 of the barrier cuffs 300 to be lifted from and separate away from the wearer facing surface 132 of the chassis 102, such as shown in FIGS. 4, 5A, and 5B. The tension of the cuff elastic material 328 may also help to hold the barrier cuffs 300 in contact with a wearer's body when wearing an absorbent article. It is to be appreciated that the cuff elastic material 328 may be configured in various ways, such as for example, one or more of an elastic film, an elastic ribbon, or an elastic strand. For example, as shown in FIGS. 3, 4, 5A, and 5B, the barrier cuffs 300 may comprise one or more cuff elastic strands 330 bonded between the first portion 324 and the second portion 326 of the cuff substrate 320. In some configurations, the barrier cuffs 300 may comprise two or more cuff elastic strands 330. In some configurations, the barrier cuffs 300 may comprise three cuff elastic strands 330. In some configurations, an inner most cuff elastic strand 330 may be positioned adjacent the distal edge 304 of the barrier cuff 300, wherein an inner most cuff elastic strand may be positioned from about 0.5 mm to about 3 mm from the distal edge 304, specifically reciting all 0.5 mm increments within the above-recited ranges and all ranges formed therein or thereby. In some configurations, an inner most cuff elastic strand may positioned greater than about 3 mm from the distal edge 304. It is also to be appreciated that the cuff elastic strands 330 may be separated from each other by various distances. For example, in some configurations, cuff elastic strands 330 may be spaced apart from each other from about 0.5 mm to about 8 mm, specifically reciting all 0.5 mm increments within the above-recited ranges and all ranges formed therein or thereby. It is to be appreciated that the barrier cuffs 300 may comprise cuff elastic strands 330 that are equally spaced from each other or are not equally spaced from each other.

In some configurations, the barrier cuffs 300 may comprise one or more inwardly positioned cuff elastic strands 330a and one or more outwardly positioned elastic strands 330b. For example, as shown in FIG. 3A, inwardly positioned elastic strands may be positioned adjacent the distal edge 304 of the barrier cuff 300, as discussed above, and the outwardly positioned elastic strands 330b may be spaced apart from the inwardly positioned elastic strands and the distal edge 304 of the barrier cuff 300 and may be positioned adjacent proximal bond regions 302. In some configurations, the outwardly positioned elastic strands 330b may be positioned from about 15 mm to about 30 mm from the distal edge 304, specifically reciting all 0.5 mm increments within the above-recited ranges and all ranges formed therein or thereby.

It is further to be appreciated that the cuff elastic strands 330 may be configured in various ways and with various decitex values. For example, in some configurations may comprise cuff elastic strands 300 that are from about 10 decitex to about 900 decitex, specifically reciting all 1 decitex increments within the above-recited ranges and all ranges formed therein or thereby. The cuff elastic strands 330 may also be pre-strained prior to joining the cuff elastic strand 330 with the cuff substrate layer 320. In some configurations, the cuff elastic strands 330 may be pre-strained from about 75 to about 300%, specifically reciting all 1% increments within the above-recited range and all ranges formed therein or thereby. It is to be appreciated that the barrier cuffs 300 may comprise cuff elastic strands 330 having the same or different pre-strain values and/or decitex.

With continued reference to FIG. 3, the barrier cuffs 300 may comprise an elasticated length 332 wherein the cuff elastic strands 330 are bonded with the cuff substrate 320 under tension. The barrier cuffs 300 may comprise an unelasticated length 334 wherein the cuff elastic strands 330 are not present or are not bonded with the cuff substrate 320 under tension. The elasticated length 332 of the barrier cuff 300 may extend longitudinally between a first unelasticated length 334a and a second unelasticated length 334b. In some configurations, the first unelasticated length 334a may extend from the first lateral end edge 144 of the chassis 102 longitudinally inward beyond the first end bond region 310a. In some configurations, the second unelasticated length 334b may extend from the second lateral end edge 146 of the chassis 102 longitudinally inward beyond the second end bond region 310b.

The leg gasketing system 156 may also be configured to comprise leg cuffs 336 in addition to the barrier cuffs 300. As discussed in more detail below, the leg cuffs 336 may be elasticated and may help hold the chassis 102 against a wearer's skin during wear. Leg cuffs 336 may also be adapted to help provide a covering to reduce the visibility of exudates through the barrier cuffs 300 and to provide a secondary structural feature to help prevent additional leaks in the event bodily exudates escaped beyond the barrier cuffs 300. As shown in FIGS. 4-6B, the leg gasketing system 156 may comprise leg cuffs 336 connected with the chassis 102. In some configurations, the leg cuffs 336 may be positioned entirely laterally outward from the proximal bond regions 302 on the wearer facing surface 132 of the chassis 102. In some configurations, the leg cuffs 336 may extend laterally outside the first and second side edges 128, 130 of the chassis 102. Various leg cuff configurations are disclosed in U.S. Pat. Nos. 3,860,003; 4,695,278; 4,704,115; 4,795,454; 4,909,803; and 8,939,957; U.S. Patent Publication No. 2009/0312730 A1; and PCT Patent Publication Nos. WO2022/183432 A1 and WO2022/183962 A1, all of which are incorporated by reference herein.

As discussed above, the barrier cuff separation distance BCS may be decreased when the cuff height CH is increased, and a relatively small the barrier cuff separation distance BCS may increase the likelihood of leaks when a wearer discharges bodily exudates on the wearer facing surface 306 of the barrier cuffs 300. In turn, the separation distance LS between the proximal bond regions 302 may be increased to help maintain a desired barrier cuff separation distance BCS when utilizing barrier cuffs 300 with relatively large cuff heights CH. However, the separation distance LS between the proximal bond regions may be limited by the chassis width CW and may be further limited when the leg gasketing system 156 comprises leg cuffs 336 laterally outboard of the proximal bond regions 302. In some configurations, such limitations may be mitigated by positioning the leg cuffs 336 partially or entirely on the garment facing surface 134 of the chassis 102, such as on the backsheet 136.

FIGS. 4 and 5A-6B show an example of the leg gasketing system 156 that may comprise a first leg cuff 336a and a second leg cuff 336b laterally separated from the first leg cuff 336a. The first leg cuff 336a may be partially defined by the first portion 324 of the cuff substrate 320 of the first barrier cuff 300a that extends laterally outward from the first proximal bond region 302a. The first portion 324 of the cuff substrate 320 may extend from the first proximal bond region 302a to a second fold line 338 where the cuff substrate 320 may be folded or wrapped around the first side edge 128 of the chassis 102. A third portion 340 of the cuff substrate 320 of the first leg cuff 300a may extend laterally inward from the second fold line 338 and may be bonded with the garment facing surface 134 of the backsheet 136. Similarly, the second leg cuff 336b may be defined by the first portion 324 of the cuff substrate 320 of the second barrier cuff 300b that extends laterally outward from the second proximal bond region 302b. The first portion 324 of the cuff substrate 320 may extend from the second proximal bond region 302b to a second fold line 338 where the cuff substrate 320 may be folded or wrapped around the second side edge 130 of the chassis 102. A third portion 340 of the cuff substrate 320 of the second leg cuff 336b may extend laterally inward from the second fold line 338 and may be bonded with the garment facing surface 134 of the backsheet 134. In some configurations, the third portion 340 of the cuff substrate 320 may extend laterally inward from the second fold line 338 from about 10 mm to about 50 mm, specifically reciting all 0.5 mm increments within the above-recited ranges and all ranges formed therein or thereby. Although the leg cuffs 336 described herein may be formed with a single cuff substrate 320 that also forms the barrier cuffs 300, it is to be appreciated that the leg cuffs 336 may be formed additional cuff substrates.

As previously mentioned, the leg cuffs 336 may be elasticated and thus may comprise a leg elastic material 342 bonded with the cuff substrate 320 under tension. As such, the tension of the leg elastic material 342 may be configured so help hold the chassis 102 against the wearer's body when wearing an absorbent article. It is to be appreciated that the leg elastic material 342 may be configured in various ways, such as for example, one or more of an elastic film, an elastic ribbon, or an elastic strand. For example, as shown in FIGS. 4, 5A, and 5B, the leg cuffs 336 may comprise one or more leg elastic strands 344 bonded with the third portion 340 of the cuff substrate 320. In some configurations, the leg cuffs 336 may comprise two or more leg elastic strands 344. In some configurations, the leg cuffs 336 may comprise four leg elastic strands 344. In some configurations, an outer most leg elastic strand 344 may be positioned adjacent the second fold line 338 of the cuff substrate 320, wherein outer most leg elastic strand may be positioned from about 0.5 mm to about 3 mm from the second fold line 338, specifically reciting all 0.5 mm increments within the above-recited ranges and all ranges formed therein or thereby. It is also to be appreciated that the leg elastic strands 344 may be separated from each other by various distances. For example, in some configurations, leg elastic strands 344 may be spaced apart from each other from about 0.5 mm to about 8 mm, specifically reciting all 0.5 mm increments within the above-recited ranges and all ranges formed therein or thereby. It is to be appreciated that the leg cuffs 336 may comprise leg elastic strands 344 that are equally spaced from each other or are not equally spaced from each other.

It is further to be appreciated that the leg elastic strands 344 may be configured in various ways and with various decitex values. For example, in some configurations may comprise leg elastic strands 344 from about 10 decitex to about 900 decitex, specifically reciting all 1 decitex increments within the above-recited ranges and all ranges formed therein or thereby. The leg elastic strands 344 may also be pre-strained prior to joining the cuff elastic strand to the cuff substrate layer. In some configurations, the leg elastic strands 344 may be pre-strained from about 75% to about 300%, specifically reciting all 1% increments within the above-recited range and all ranges formed therein or thereby. It is to be appreciated that the leg cuffs 336 may comprise leg elastic strands 344 having the same or different pre-strain values and/or decitex. It is also to be appreciated that the leg elastic strands 344 and the cuff elastic strands 330 may comprise the same or different decitex and/or pre-strain values. In addition, the leg elastic strands 344 and the cuff elastic strands 330 may comprise the same or different spacing.

As shown in FIGS. 4, 5A, and 5B, at least one leg elastic strand 344 may be bonded between the first portion 324 and the third portion 340 of the cuff substrate 320. In some configurations, at least one leg elastic strand 344 may be bonded between the third portion 340 of the cuff substrate 320 and the backsheet 136. In some configurations, the third portion 340 of the cuff substrate 320 may extend laterally inward from the second fold line 338 to a third fold line 346 where a fourth portion 348 of the cuff substrate 320 is positioned in a facing relationship with the third portion 340 of the cuff substrate 320. As such, the fourth portion 348 of the cuff substrate 320 may be positioned between the third portion 340 of the cuff substrate 320 and the backsheet 136. In turn, at least one leg elastic strand 344 may bonded between the third portion 340 and the fourth portion 348 of the cuff substrate 320.

As previously mentioned, it is to be appreciated that the leg gasketing systems 156 herein may comprise various different leg cuff configurations. For example, FIGS. 4A, 5AA, 5BA, 6AA, and 6BA illustrate another example configuration of leg cuffs 156 configured without the above described third fold line 346 and fourth portion 348. As such, in FIGS. 4A, 5AA, 5BA, 6AA, and 6BA, the at least one leg elastic strand 344 may be bonded between the first portion 324 and the third portion 340 of the cuff substrate 320 and/or may be bonded between the third portion 340 of the cuff substrate 320 and the backsheet 136.

As discussed above, a wearer that sleeps on their side may cause the pee point to shift laterally from a central location of the absorbent article 100 or chassis 102 laterally to a side location of the absorbent article 100 or chassis 102. It is to be appreciated that the first void regions 312a and/or the second void regions 312b positioned adjacent side edges of the chassis 102 in the waist regions 116, 118 are adapted to accept and hold bodily exudates discharged from a wearer at a pee point that may have shifted laterally to a side location. In turn, such exudates may be held in the void regions 312 while being absorbed by the absorbent assembly 140. The Cuff Leakage Test Method herein may be used to measure the quantity of simulated urine that escapes from a diaper when positioned in a way so as to generally mimic a wearer lying on one's side. As demonstrated below in Table 1, a comparison of absorbent article articles tested with Cuff Leakage Test Method, and in particular, the “Leaked Saline Amount” values show significant improvement in the effectiveness of the barrier cuffs 300 herein to help prevent unintended leakage during discharges of exudates in situations where a wearer of an absorbent article is sleeping or lying on their side, which results in a lateral side shift the pee point. It is to be appreciated that the “Leaked Saline Amount” may vary over a range of values for some configurations of absorbent articles in accordance with the present disclosure, for example, the “Leaked Saline Amount” may be from about 0.5 grams to about 3.0 grams in some configurations, specifically reciting all 0.1 gram increments within the above-recited ranges and all ranges formed therein or thereby.

TABLE 1

| | Current Market | Inventive |
|---|---|---|
| | Product Sample | Example |
| Dry Pad Weight(g) Avg | 38.02 | 43.82 |
| Wet Pad Weight(g)Avg | 80.34 | 104.78 |
| Leaked Saline Amount (g) Avg | 20.48 | 1.22 |
| Leaked Saline Amount (g) Stdev | 1.06 | 1.496 |
| Delivered Saline Volume(g)Avg | 63.42 | 62.18 |

TABLE 1
Current Market Product Sample Description:

Current market Ninjamas ™ S/M as commercially available in USA in
April 2024 with a leg gasketing system with the following aspects:
Barrier cuff material: 13 gsm SSMMSS nonwoven;
2 cuff elastic strands;
Cuff Height (CH): 32 mm; and
No triangularly shaped void regions positioned adjacent side edges of
the chassis in the front and rear waist regions.

TABLE 1
Inventive Example Description:

The inventive example diaper pant comprises a leg gasketing system
with the following aspects:
Barrier cuff material: 13 gsm SSMMSS nonwoven;
3 cuff elastic strands;
First Cuff Height (CH1): 51 mm;
Second Cuff Height (CH2): 51 mm;
Center Cuff Height (CHC): 40 mm; and
Triangularly shaped first void regions and second void regions positioned
adjacent side edges of the chassis in the front and rear waist regions.

It is to be appreciated that the leg gasketing systems 156 herein provide for barrier cuffs 300 that may be bonded with the chassis 102 in proximal bond regions 302 configured to allow for increased cuff heights CH while at the same time helping to mitigate wearer entanglement during donning. In addition, the end bond regions 310 of the barrier cuffs 300 configured to provide void regions 312 between the barrier cuffs 300 and the chassis 102 help prevent urine from leaking outside the article while the urine is being absorbed by the absorbent assembly 140. Further, the leg gasketing systems 156 may also be configured with leg cuffs 336 bonded with the garment facing surface 134 of the backsheet 136 to help provide for increased flexibility with regard to laterally outboard positioning of proximal bond regions 302. As such, barrier cuffs 300 may comprise relatively large cuff heights while at the same time maintaining lateral spacing between the barrier cuffs 300 to help avoid excessive coverage of the absorbent core 142 by the barrier cuffs 300.

### Hydrostatic Head Test

Hydrostatic head is tested using a TexTest FX3000 Hydrostatic Head Tester (available from Advanced Testing Instruments, Greer, SC) with a custom made 1.5 cm² circular measurement port (also available from Advanced Testing Instruments). Two annular sleeve rings, the same dimensions as the gaskets around the measurement ports, are cut from the standard protective sleeves for fine nonwovens (part FX3000-NWH, available from Advanced Testing Instruments). The sleeve rings are then adhered with two-sided adhesive tape to the sample facing surfaces of the upper and lower gaskets of the TexTest instrument to protect the specimen during clamping. Standardize the instrument according to the manufacturer's procedures. All testing is performed in a room maintained at about 23° C.±2C.° and about 50%±2% relative humidity.

Precondition the articles at about 23° C.±2C.° and about 50%±2% relative humidity for two hours prior to testing. To obtain a specimen, lay the article stretched flat on a bench, body facing surface upward, and measure the total longitudinal length of the article. Select a testing site on the barrier cuff (300) at the longitudinal midpoint of the article. Using scissors cut a test specimen 70 mm long by the entire height of the barrier cuff (300) centered at the longitudinal midpoint on both the left and right sides. Any elastic members are removed.

Place the specimen centered over the port of the upper test head. The specimen should completely cover the port with the surface corresponding to the outward-facing surface of the cuff directed toward the port (inner-facing surface will then be facing the water). Gently extend the specimen taut in its longitudinal direction so that the specimen lies flat against the upper test plate. Adhesive tape is applied to secure the specimen to the test plate in its extended state for testing. Tape should not cover any portion of the measurement port.

Fill the TexTest syringe with distilled water, adding the water through the measurement port of the lower test plate. The water level should be filled to the top of the lower gasket. Mount the upper test head onto the instrument and lower the test head to make a seal around the specimen. The test speed is set to 3 mbar/min for samples that have a hydrostatic head of 50 mbar or less and a speed of 60 mbar/min for samples with a hydrostatic head above 50 mbar. Start the test and observe the specimen surface to detect water droplets penetrating the surface. The test is terminated when one drop is detected on the surface of the specimen or the pressure exceeds 200 mbar. Record the pressure to the nearest 0.5 mbar or record as >200 mbar if there was no penetration detected.

A total of five identical articles (10 barrier cuff (300) specimens) are analyzed and their hydrostatic head results recorded. Calculate and report the average hydrostatic head report to the nearest 0.1 mbar.

### Cuff Leakage Test Method

The Cuff Leakage Test Method is used to measure the quantity of simulated urine that escapes from a diaper when positioned in a way so as to generally mimic a wearer lying on one's side. The crotch panel specimen of a diaper sample of interest is isolated and fastened to a rigid board oriented at an angle. A pump is used to deliver a predetermined volume of physiological saline at a predetermined rate to a specified location at the surface of the diaper, and any saline that escapes the diaper is captured. Based on dry and wet masses, delivered saline mass is confirmed and leaked saline mass is quantified. At least five replicate specimens of a diaper sample of interest are measured, and the arithmetic mean of the masses are reported as the Delivered Saline Amount and Leaked Saline Amount, respectively, both in grams. Samples are stored at 23° C.±3° C. and 50%±2% relative humidity for at least two hours prior to analysis, and all laboratory work is done under these same conditions.

### Sample Preparation

For a given diaper sample of interest, at least five replicate specimens are prepared. For each specimen, the crotch panel (also referred to as a chassis 102 as described herein) is isolated as much as possible by delaminating, cutting away, or otherwise removing any belt panels or side panels at either end of the crotch panel. All barrier cuffs 300 and leg cuffs 336 are left entirely intact. On each specimen, the “pee point” is identified and marked. This point is found by measuring the point along the lateral centerline (longitudinal axis) of the article that is 25% of the total crotch panel length from the waist edge.

### Apparatus

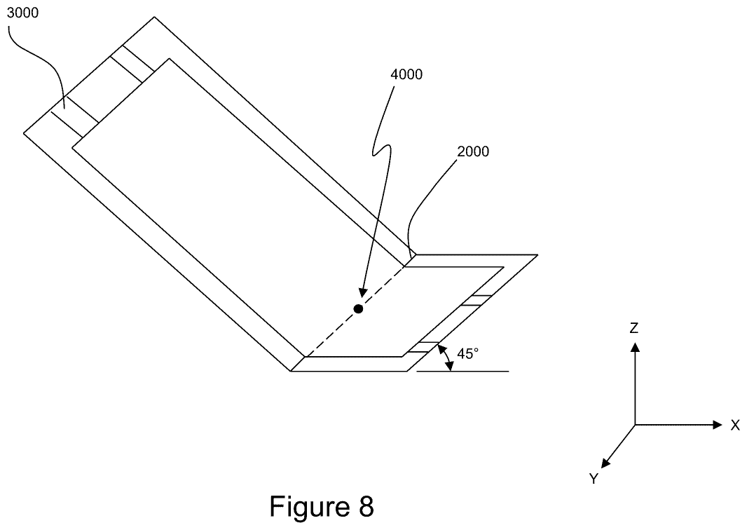

An angled stretch board 1000 is used which is at least as long and wide as is each of the like specimen crotch panels of the diaper of interest as shown in FIG. 7. The stretch board is fashioned from water-resistant, rigid material such as ¼ ″ polycarbonate or acrylic sheet. The stretch board contains an angled crease at 30±3° along a laterally oriented crease line 2000 such that it is possible to affix the entire crotch panel to the stretch board flat, with the pee point 4000 coincident with the crease line 2000. Adhesive hook material strips 3000 are affixed to the stretch board so as to enable the crotch panel specimen to be held flat and securely against the flat surfaces of the stretch board. In addition, edge-mounted clamps may optionally be used to ensure that the longitudinally running lateral edges of the crotch panel remain flat even when loaded with saline.

### Procedure

A crotch panel specimen of the diaper of interest is first weighed (recorded to nearest 0.01 g) and then mounted flat and outstretched on the stretch board such that the pee point is coincident with the angled crease line 2000 on the stretch board. The stretch board is then lifted at least 20 cm above the work surface and held for the duration of the procedure in a fixed position such that the plane of the stretch board corresponding to the front portion of the crotch panel forms a 45±3° angle to horizontal (the xy plane) and such that the lateral edge of the diaper in this front portion remains horizontal as shown in FIG. 8. (This may be accomplished via a range of suitable means, such as attaching the stretch board releasably to a clamping structure or fashioning a cradle into which the stretch board may be rested to enable this fixed orientation. The stretch board may even be fashioned so as to contain an integral stand base that assures this orientation when placed on a horizontal work surface. Any other creases present in the stretch board used must allow the front portion of the crotch panel to be affixed in a single plane as described and above all other portions of the crotch panel to be oriented vertically at or above the same elevation z as the front panel as shown in FIG. 8.)

Beneath the raised stretch board is located a pan to catch fluid, and sheets of paper towel may optionally be placed in the pan to absorb fluid and reduce splashing. The dry crotch panel mass and dry pan mass (which may optionally contain dry paper towel) are captured and recorded to the nearest 0.01 g.

A pump (such as a suitable peristaltic or piston pump) capable of delivering 60 mL at 15 mL/s is used to deliver ambient-temperature physiological saline (0.9% sodium chloride in deionized water) to the crotch panel mounted as described above. A tube with inner diameter 2 mm or less is interfaced with the pump at one end and oriented at the other end via suitable means (e.g. using a ring stand and clamp) such that the exit orifice is at the pee point of the crotch panel. 60 mL of saline is then delivered at 15 mL/s, which generally results in some portion of saline being retained by the crotch panel and at least some portion of fluid spilling from the tilted crotch panel into the catch pan. (While the intention is to deliver 60 mL of saline, it is understood that delivered quantity of saline may slightly vary depending on pump characteristics. The masses captured below generally account for these small deviations.)

Immediately following the delivery of saline (and absorbance of visible free saline has completed), the wet crotch panel mass and the mass of the pan (containing any fluid captured and the same paper towel included in the dry mass) are captured and recorded to the nearest 0.01 g.

### Calculations and Reporting

For each crotch panel specimen measured, the difference between the sum of the wet crotch panel mass and wet pan mass and the sum of the dry crotch panel mass and the dry pan mass is the delivered saline amount for that specimen, recorded to the nearest 0.01 g. For each crotch panel specimen measured, the difference between the wet pan mass and the dry pan mass is the leaked saline amount for that specimen, recorded to the nearest 0.01 g. The arithmetic mean of the delivered saline amounts for all crotch panel specimens measured is calculated and reported to the nearest 0.01 g as the Delivered Saline Amount for the diaper sample of interest. The arithmetic mean of the leaked saline amount for all crotch panel specimens measured is calculated and reported to the nearest 0.01 g as the Leaked Saline Amount for the diaper sample of interest.

### COMBINATIONS

A1. An absorbent article comprising: a first waist region and a second waist region longitudinally separated from the first waist region by a crotch region; a chassis comprising a topsheet, a backsheet; and an absorbent core positioned between the topsheet and the backsheet, wherein the chassis further comprises a first lateral end edge in the first waist region longitudinally separated from a second lateral end edge in second waist region and comprising a longitudinal length CL of from about 400 mm to about 600 mm extending between the first lateral end edge and the second lateral end edge, and wherein the chassis further comprises a first longitudinal side edge laterally separated from a second longitudinal side edge; a leg gasketing system comprising a first barrier cuff and a second barrier cuff laterally separated from the first barrier cuff; a proximal bond region connecting the first barrier cuff with the chassis adjacent the first longitudinal side edge, the proximal bond region extending longitudinally for substantially the same length CL as the chassis, the first barrier cuff extending laterally inward toward the second barrier cuff from the proximal bond region to a distal edge; and wherein the first barrier cuff comprises a central segment extending longitudinally between a first segment and a second segment, the first segment extending longitudinally between the first end bond region and the central segment, and the second segment extending longitudinally between the second end bond region and the central segment, wherein the first barrier cuff is not bonded with the chassis between the proximal bond region and the distal edge in the first segment, the central segment, and the second segment, and wherein the first segment of the first barrier cuff comprises: a first cuff height defined by a distance between the proximal bond region and the distal edge, the second segment comprises a second cuff height defined by a distance between the proximal bond region and the distal edge, and the central segment comprises a central cuff height defined by a distance between the proximal bond region and the distal edge, and wherein the central cuff height is less than the first height and the second cuff height.
A2. The absorbent article of paragraph A1, further comprising: a first end bond region adjacent the first lateral end edge of the chassis, the first end bond region connecting the first barrier cuff with the chassis, wherein the first end bond region extends longitudinally inward from the first lateral end edge and extends laterally inward from the proximal bond region toward the distal edge to define a first void region where the first barrier cuff is not bonded with the chassis between the first end bond region and the proximal bond region, wherein the first void region defines a first void volume adapted to accept bodily exudates; and a second end bond region adjacent the second lateral end edge of the chassis, the second end bond region connecting the first barrier cuff with the chassis, wherein second end bond region extends longitudinally inward from the second lateral end edge and extends laterally inward from the proximal bond region toward the distal edge to define a second void region where the first barrier cuff is not bonded with the topsheet between the second end bond region and the proximal bond region, wherein the second void region defines a second void volume adapted to accept bodily exudates.
A3. The absorbent article of either paragraph A1 or A2, wherein the first barrier cuff comprises a substrate folded along a fold line to position a first portion of the substrate in a facing relationship with a second portion of the substrate.
A4. The absorbent article of paragraph A3, wherein the fold line defines the distal edge of the first barrier cuff.
A5. The absorbent article of paragraph A3, wherein the second portion is bonded between the first portion of the substrate and the chassis in the proximal bond region.
A6. The absorbent article of paragraph A3, wherein the first barrier cuff further comprises at least one cuff elastic strand bonded between the first portion and the second portion adjacent the fold line.
A7. The absorbent article of paragraph A6, wherein the at least one cuff elastic strand comprises two or more cuff elastic strands.
A8. The absorbent article of paragraph A7, wherein the two or more cuff elastic strands are spaced apart from each other by at least 2 mm.
A9. The absorbent article of any of paragraphs A3-A8, wherein the substrate is also bonded with the backsheet to define a first leg cuff.
A10. The absorbent article of paragraph A9, wherein the first portion the substrate extends laterally outward from the proximal bond region to a second fold line, and a third portion of the substrate extends laterally inward from the second fold like and is bonded with a garment facing surface of the backsheet to further define the first leg cuff.
A11. The absorbent article of paragraph A10, further comprising at least one leg elastic strand bonded between the third portion of the substrate and the backsheet.
A12. The absorbent article of paragraph A11, wherein the at least one leg elastic strand comprises two or more leg elastic strands.
A13. The absorbent article of paragraph A12, wherein the two or more leg elastic strands are spaced apart from each other by at least 2 mm.
A14. The absorbent article of paragraph A11, wherein the third portion of the substrate extends laterally inward from the second fold line to a third fold line, and wherein a fourth portion of the substrate extends laterally outward from the third fold line in a facing relationship with third portion of the substrate.
A15. The absorbent article of paragraph A14, wherein the at least one leg elastic strand is bonded between the third portion and the fourth portion of the substrate.
A16. The absorbent article of paragraph A15, wherein the fourth portion of the substrate is positioned between the third portion and the backsheet.
A17. The absorbent article of any of paragraphs A11-A16, wherein the at least one cuff elastic strand comprises a first decitex and the at least one leg elastic strand comprises a second decitex, wherein the first decitex and the second decitex are greater than about 400 decitex.
A18. The absorbent article of paragraph A17, wherein the first decitex is not equal to the second decitex.
A19. The absorbent article of paragraph A3, wherein the substrate comprises a nonwoven.
A20. The absorbent article of paragraph A19, wherein the nonwoven comprises a hydrostatic head of greater than about 2 mbar and less than about 200 mbar.
A21. The absorbent article of any of paragraphs A1-A20, wherein the distal edge of the first barrier cuff comprises an elasticated length extending longitudinally between a first unelasticated length and a second unelasticated length.
A22. The absorbent article of paragraph A21, wherein the first unelasticated length extends from the first lateral end edge of the chassis longitudinally inward beyond the first end bond region.
A23. The absorbent article of any of paragraphs A1-A22, wherein at least one of the proximal bond region, the first end bond region, and the second end bond region comprises adhesive.
A24. The absorbent article of any of paragraphs A1-A23, wherein at least one of the proximal bond region, the first end bond region, and the second end bond region comprises mechanical bonds.
A25. The absorbent article of paragraph A24, wherein the mechanical bonds comprise a plurality of intermittent bonds.
A26. The absorbent article of any of paragraphs A1-A25, further comprising a second proximal bond region connecting the second barrier cuff with the chassis adjacent the second longitudinal side edge, the second barrier cuff extending laterally inward toward the first barrier cuff from the second proximal bond region to a distal edge, wherein the distal edge of the first barrier cuff and the distal edge of the second barrier cuff are separated by at least about 80 mm.
A27. The absorbent article of any of paragraphs A1-A26, further comprising a first belt connected with the chassis in the first waist region.
A28. The absorbent article of paragraph A27, further comprising a second belt connected with the chassis in the second waist region.
A29. The absorbent article of paragraph A28, wherein opposing end regions of the first belt are connected with opposing end regions of the second belt.
A30. The absorbent article of any of paragraphs A1-A29, further comprising fasteners connected with the chassis in the first waist region and adapted to refastenably connect with the second waist region.
A31. The absorbent article of any of paragraphs A1-A30, wherein the first and second cuff heights are at least about 45 mm.
A32. The absorbent article of any of paragraphs A1-A31, wherein the distal edge of the first barrier cuff is bonded with the chassis in at least one of the first end bond region and the second end bond region.
A33. The absorbent article of any of paragraphs A1-A32, wherein the first barrier cuff comprises one or more inwardly positioned cuff elastic strands and one or more outwardly positioned cuff elastic strands, wherein the one or more inwardly positioned cuff elastic strands are positioned adjacent the distal edge and wherein the one or more outwardly positioned cuff elastic strands are positioned adjacent the proximal bond region.
A34. The absorbent article of any of paragraphs A1-A33, wherein at least one of the first end bond region and the second end bond region comprises a plurality of discrete bonds, wherein a relatively constant number of bonds are aligned in a lateral direction throughout a longitudinal length of the end bond region, and wherein a variable number of bonds are aligned in a longitudinal direction throughout a lateral width of the end bond region.
B1. Gender specific absorbent articles comprising: a first absorbent article configured for a wearer of a first gender and a second absorbent article configured for a wearer of a second gender; wherein the first absorbent article and the second absorbent article each comprise: a first waist region and a second waist region longitudinally separated from the first waist region by a crotch region; a chassis comprising a topsheet, a backsheet; and an absorbent core positioned between the topsheet and the backsheet, wherein the chassis further comprises a first lateral end edge in the first waist region longitudinally separated from a second lateral end edge in second waist region and comprising a longitudinal length CL of from about 400 mm to about 600 mm extending between the first lateral end edge and the second lateral end edge, and wherein the chassis further comprises a first longitudinal side edge laterally separated from a second longitudinal side edge; a leg gasketing system comprising a first barrier cuff and a second barrier cuff laterally separated from the first barrier cuff; a proximal bond region connecting the first barrier cuff with the chassis adjacent the first longitudinal side edge, the proximal bond region extending longitudinally for substantially the same length CL as the chassis, the first barrier cuff extending laterally inward toward the second barrier cuff from the proximal bond region to a distal edge; a first end bond region adjacent the first lateral end edge of the chassis, the first end bond region connecting the first barrier cuff with the chassis, wherein the first end bond region extends longitudinally inward from the first lateral end edge and extends laterally inward from the proximal bond region toward the distal edge to define a first void region where the first barrier cuff is not bonded with the chassis between the first end bond region and the proximal bond region, wherein the first void region defines a first void volume adapted to accept bodily exudates; a second end bond region adjacent the second lateral end edge of the chassis, the second end bond region connecting the first barrier cuff with the chassis, wherein second end bond region extends longitudinally inward from the second lateral end edge and extends laterally inward from the proximal bond region toward the distal edge to define a second void region where the first barrier cuff is not bonded with the topsheet between the second end bond region and the proximal bond region, wherein the second void region defines a second void volume adapted to accept bodily exudates; and wherein the first barrier cuff comprises a central segment extending longitudinally between a first segment and a second segment, the first segment extending longitudinally between the first end bond region and the central segment, and the second segment extending longitudinally between the second end bond region and the central segment, wherein the first barrier cuff is not bonded with the chassis between the proximal bond region and the distal edge in the first segment, the central segment, and the second segment, and wherein the first segment of the first barrier cuff comprises: a first cuff height defined by a distance between the proximal bond region and the distal edge, the second segment comprises a second cuff height defined by a distance between the proximal bond region and the distal edge, and the central segment comprises a central cuff height defined by a distance between the proximal bond region and the distal edge, and wherein the central cuff height is less than the first height and the second cuff height; and wherein the first cuff height and the second cuff height of the first absorbent article are larger than the first cuff height and the second cuff height of the second absorbent article.
B2. The gender specific absorbent articles of paragraph B2, wherein the first gender is a male gender and the second gender is a female gender.

### Bio-Based Content for Components

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. Appl. Publ. No. 2019/0192723, published on Jun. 27, 2019.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a first waist region and a second waist region longitudinally separated from the first waist region by a crotch region;
a chassis comprising a topsheet, a backsheet; and an absorbent core positioned between the topsheet and the backsheet, wherein the chassis further comprises a first lateral end edge in the first waist region longitudinally separated from a second lateral end edge in second waist region and comprising a longitudinal length CL of from about 400 mm to about 600 mm extending between the first lateral end edge and the second lateral end edge, and wherein the chassis further comprises a first longitudinal side edge laterally separated from a second longitudinal side edge;
a leg gasketing system comprising a first barrier cuff and a second barrier cuff laterally separated from the first barrier cuff;
a proximal bond region connecting the first barrier cuff with the chassis adjacent the first longitudinal side edge, the proximal bond region extending longitudinally for substantially the same length CL as the chassis, the first barrier cuff extending laterally inward toward the second barrier cuff from the proximal bond region to a distal edge;
a first end bond region adjacent the first lateral end edge of the chassis, the first end bond region connecting the first barrier cuff with the chassis, wherein the first end bond region extends longitudinally inward from the first lateral end edge and extends laterally inward from the proximal bond region toward the distal edge to define a first void region where the first barrier cuff is not bonded with the chassis between the first end bond region and the proximal bond region, wherein the first void region defines a first void volume adapted to accept bodily exudates;
a second end bond region adjacent the second lateral end edge of the chassis, the second end bond region connecting the first barrier cuff with the chassis, wherein second end bond region extends longitudinally inward from the second lateral end edge and extends laterally inward from the proximal bond region toward the distal edge to define a second void region where the first barrier cuff is not bonded with the chassis between the second end bond region and the proximal bond region, wherein the second void region defines a second void volume adapted to accept bodily exudates; and
wherein the first barrier cuff comprises a central segment extending longitudinally between a first segment and a second segment, the first segment extending longitudinally between the first end bond region and the central segment, and the second segment extending longitudinally between the second end bond region and the central segment, wherein the first barrier cuff is not bonded with the chassis between the proximal bond region and the distal edge in the first segment, the central segment, and the second segment, and
wherein the first segment of the first barrier cuff comprises: a first cuff height defined by a distance between the proximal bond region and the distal edge, the second segment comprises a second cuff height defined by a distance between the proximal bond region and the distal edge, and the central segment comprises a central cuff height defined by a distance between the proximal bond region and the distal edge, and
wherein the central cuff height is less than the first height and the second cuff height.

2. The absorbent article of claim 1, wherein the first barrier cuff comprises a substrate folded along a fold line to position a first portion of the substrate in a facing relationship with a second portion of the substrate.

3. The absorbent article of claim 2, wherein the fold line defines the distal edge of the first barrier cuff.

4. The absorbent article of claim 2, wherein the second portion is bonded between the first portion of the substrate and the chassis in the proximal bond region.

5. The absorbent article of claim 2, wherein the first barrier cuff further comprises at least one cuff elastic strand bonded between the first portion and the second portion adjacent the fold line.

6. The absorbent article of claim 5, wherein the at least one cuff elastic strand comprises two or more cuff elastic strands.

7. The absorbent article of claim 6, wherein the two or more cuff elastic strands are spaced apart from each other by at least 2 mm.

8. The absorbent article of claim 5, wherein the substrate is also bonded with the backsheet to define a first leg cuff.

9. The absorbent article of claim 8, wherein the first portion the substrate extends laterally outward from the proximal bond region to a second fold line, and a third portion of the substrate extends laterally inward from the second fold line and is bonded with a garment facing surface of the backsheet to further define the first leg cuff.

10. The absorbent article of claim 9, further comprising at least one leg elastic strand bonded between the third portion of the substrate and the backsheet.

11. The absorbent article of claim 10, wherein the at least one leg elastic strand comprises two or more leg elastic strands.

12. The absorbent article of claim 11, wherein the two or more leg elastic strands are spaced apart from each other by at least 2 mm.

13. The absorbent article of claim 10, wherein the third portion of the substrate extends laterally inward from the second fold line to a third fold line, and wherein a fourth portion of the substrate extends laterally outward from the third fold line in a facing relationship with third portion of the substrate.

14. The absorbent article of claim 13, wherein the at least one leg elastic strand is bonded between the third portion and the fourth portion of the substrate.

15. The absorbent article of claim 14, wherein the fourth portion of the substrate is positioned between the third portion and the backsheet.

16. The absorbent article of claim 10, wherein the at least one cuff elastic strand comprises a first decitex and the at least one leg elastic strand comprises a second decitex, wherein the first decitex and the second decitex are greater than about 400 decitex.

17. The absorbent article of claim 16, wherein the first decitex is not equal to the second decitex.

18. The absorbent article of claim 2, wherein the substrate comprises a nonwoven.

19. The absorbent article of claim 18, wherein the nonwoven comprises a hydrostatic head of greater than about 2 mbar and less than about 200 mbar.

20. The absorbent article of claim 1, wherein the distal edge of the first barrier cuff comprises an elasticated length extending longitudinally between a first unelasticated length and a second unelasticated length.

21. The absorbent article of claim 20, wherein the first unelasticated length extends from the first lateral end edge of the chassis longitudinally inward beyond the first end bond region.

22. The absorbent article of claim 1, wherein at least one of the proximal bond region, the first end bond region, and the second end bond region comprises adhesive.

23. The absorbent article of claim 1, wherein at least one of the proximal bond region, the first end bond region, and the second end bond region comprises mechanical bonds.

24. The absorbent article of claim 23, wherein the mechanical bonds comprise a plurality of intermittent bonds.

25. The absorbent article of claim 1, further comprising a second proximal bond region connecting the second barrier cuff with the chassis adjacent the second longitudinal side edge, the second barrier cuff extending laterally inward toward the first barrier cuff from the second proximal bond region to a distal edge, wherein the distal edge of the first barrier cuff and the distal edge of the second barrier cuff are separated by at least about 80 mm.

26. The absorbent article of claim 1, further comprising a first belt connected with the chassis in the first waist region.

27. The absorbent article of claim 26, further comprising a second belt connected with the chassis in the second waist region.

28. The absorbent article of claim 27, wherein opposing end regions of the first belt are connected with opposing end regions of the second belt.

29. The absorbent article of claim 1, further comprising fasteners connected with the chassis in the first waist region and adapted to refastenably connect with the second waist region.

30. The absorbent article of claim 1, wherein the first and second cuff heights are at least about 45 mm.

31. The absorbent article of claim 1, wherein the distal edge of the first barrier cuff is bonded with the chassis in at least one of the first end bond region and the second end bond region.

32. The absorbent article of claim 1, wherein the first barrier cuff comprises one or more inwardly positioned cuff elastic strands and one or more outwardly positioned cuff elastic strands, wherein the one or more inwardly positioned cuff elastic strands are positioned adjacent the distal edge and wherein the one or more outwardly positioned cuff elastic strands are positioned adjacent the proximal bond region.

33. The absorbent article of claim 1, wherein at least one of the first end bond region and the second end bond region comprises a plurality of discrete bonds, wherein a relatively constant number of bonds are aligned in a lateral direction throughout a longitudinal length of the end bond region, and wherein a variable number of bonds are aligned in a longitudinal direction throughout a lateral width of the end bond region.
